(19) United States
(12) Patent Application Publication
(10) Pub. No.: US 2021/0128534 A1
(43) Pub. Date: May 6, 2021

(54) DRUG DELIVERY SYSTEMS

(71) Applicant: YISSUM RESEARCH DEVELOPMENT COMPANY OF THE HEBREW UNIVERSITY OF JERUSALEM LTD, Jerusalem (IL)

(72) Inventors: Simon BENITA, Tel Aviv (IL); Taher NASSAR, Kfar Tur'an (IL); Leslie REBIBO, Jerusalem (IL); Amit BADIHI, Jerusalem (IL)

(73) Assignee: Yissum Research Development Company of the Hebrew University of Jerusalem Ltd., Jerusalem (IL)

(21) Appl. No.: 16/971,465

(22) PCT Filed: Feb. 26, 2019
(86) PCT No.: PCT/IL2019/050217

§ 371 Date: Nov. 18, 2020

Related U.S. Application Data
(60) Provisional application No. 62/635,088, filed on Feb. 26, 2018.

(51) Int. Cl. A61K 31/436; A61K 38/13; A61K 31/352; A61K 31/05; A61K 31/282; A61K 31/56; A61K 31/404; A61K 31/573; A61K 9/19; A61K 47/34; A61K 47/44; A61K 47/40; A61K 47/26; A61K 47/10; A61K 47/32; A61K 47/12

(52) U.S. Cl. CPC A61K 31/436; A61K 47/12; A61K 31/352; A61K 31/05; A61K 31/282; A61K 31/56; A61K 31/404; A61K 31/573; A61K 9/19; A61K 47/34; A61K 47/44; A61K 47/40; A61K 47/26; A61K 47/10; A61K 47/32; A61K 38/13

(57) ABSTRACT

A novel platform for manufacturing storage stable and effective drug delivery systems.

### TECHNOLOGICAL FIELD

The invention generally provides unique delivery systems, reconstituted solutions and uses thereof.

### BACKGROUND

Management of atopic dermatitis (AD) is a therapeutic challenge that comprises optimal skin care, topical therapy and systemic treatment. Topical corticosteroids (TCS) are the first-line therapeutics used for AD treatment due to their anti-inflammatory, immunosuppressive and anti-proliferative effects. However, they have many local and systemic side effects, associated with long-term therapy. Tacrolimus and pimecrolimus, show higher selectivity, higher efficiency and a better short-term safety profile in comparison to TCS. However, due to the lack of long-term safety data, a widespread off-label use and potential risks of skin cancer and lymphomas, the Pediatric Advisory of the FDA recommended a “black box” warning for these agents, limiting their usage.

Cyclosporine A (CsA) exhibits similar immunomodulatory properties as tacrolimus and pimecrolimus. CsA shows a remarkable efficacy in the treatment of a multitude of dermatological diseases when administered orally. In fact, CsA therapy is the first line short-term systemic therapy in severe AD. Indeed, long-term systemic administration of CsA is associated with serious side effects including renal dysfunction, chronic nephrotoxicity and hypertension.

Unfortunately, owing to its large molecular weight and poor water solubility, CsA penetration into skin layers following topical application is limited. Furthermore, the promise of CsA delivery into the intact skin mediated by various nanocarriers encountered little success if any.

### REFERENCES

[1] Fessi H, Puisieux F, Devissaguet J P, Ammoury N, Benita S. Nanocapsule formation by interfacial polymer deposition following solvent displacement. Int J Phar 1989; 55: R1-R4.
[2] WO 2012/101638
[3] WO 2012/101639

### GENERAL DESCRIPTION

The inventors of the technology disclosed herein have developed a novel platform for manufacturing storage stable and effective drug delivery systems that may be tailored for a variety of applications, in a variety of formulations and which may be tailored to meet one or more requirements associated with drug delivery.

The technology is based on a nanocarrier system in the form of poly lactic-co-glycolic acid (PLGA)-nanospheres (NSs) and nanocapsules (NCs) that enhance drug penetration into the skin. The carrier system is provided as freeze-dried nanoparticles (NPs) that may be incorporated in an anhydrous topical formulation and which provides improved drug skin absorption and adequate dermato-biodistribution (DBD) profiles in various skin layers, as exemplified ex vivo.

The various PLGA nanocarriers containing an active, such as CsA, were prepared according to the well-established solvent displacement method [1] and full details are presented in the experimental section below.

Thus, in most general terms, the invention provides a lyophilized solid powder formulation configured for reconstitution in a liquid carrier, which may be water-based carrier, for some of the applications disclosed herein (particularly those for immediate use), or which may be an anhydrous carrier (water free), such as a silicone-based carrier, for other applications, particularly those necessitating prolonged storage periods. The solid powder may alternatively be used as such, in a non-liquid or formulated form.

In a first aspect, the invention provides a powder comprising a plurality of PLGA nanoparticles, each nanoparticle comprising at least one non-hydrophilic material (drug or active), the powder being in the form of dry flakes, typically achievable by lyophilization.

In some embodiments, the dry powder further comprises at least one cryoprotectant, that may optionally be selected from cyclodextrin, PVA, sucrose, trehalose, glycerin, dextrose, polyvinylpyrrolidone, mannitol, xylitol and others.

In some embodiments, lyophilization is carried out in the presence of at least one cryoprotectant, that may be selected as above.

In a further aspect, the invention provides a ready-for-reconstitution powder comprising a plurality of PLGA nanoparticles, each nanoparticle comprising at least one non-hydrophilic material (drug or active). The powder may be a dry solid, as defined, yet, under some conditions and depending on the content of oils or waxy materials, the product may have a consistency of an ointment.

The invention further provides a solid dosage form of at least one non-hydrophilic drug, the dosage form being a dry powder comprising a plurality of PLGA nanoparticles, each nanoparticle comprising the at least one non-hydrophilic material (drug or active).

In some embodiments, a dry powder or a reconstituted formulation according to the invention comprises ingredients or carriers or excipients that do not cause, directly or indirectly, substantial (no more than 15-20% or 10-15% of the total population of the nanoparticles) leaching out of the at least one non-hydrophilic material from the nanoparticle in which it is contained over a period immediately after the dry powder or reconstituted formulation is manufactured or within 7 days from its manufacture.

The “at least one non-hydrophilic material” that is contained in PLGA nanoparticles of the invention is a drug or a therapeutically active agent that is water insoluble, or a drug or a therapeutically active agent that is hydrophobic, or amphiphilic in nature. In some embodiments, the at least one non-hydrophilic material is characterized by being above logP value of 1, the LogP value being an estimate of a compound overall lipophilicity and partition between the aqueous and organic liquid phases where the active ingredient has been dissolved.

In some embodiments, the at least non-hydrophilic material is selected from cyclosporine A (Cys A), tacrolimus, pimecrolimus, dexamethasone palmitate, Cannabis lipophilic extracted derivatives such as tetrahydrocannabinol (THC) and cannabidiol (CBD) (phytocannabinoids), or synthetic cannabinoids, zafirlukast, finasteride, oxaliplatin palmitate acetate (OPA) and others.

In some embodiments, the non-hydrophobic material is selected from cyclosporine A (Cys A), tacrolimus and pimecrolimus. In some embodiments, the non-hydrophobic material is cyclosporine A (Cys A) or tacrolimus or pimecrolimus or CBD or THC or finasteride or oxaliplatin palmitate acetate (OPA).

In some embodiments, the non-hydrophilic material is not cyclosporine.

Cyclosporine, shown in Formula (I), is an immunosuppressant macromolecule that interferes with the activity and growth of T cells, thereby reducing the activity of the immune system. As can be appreciated, due to its relatively large size, topical delivery of cyclosporine has proven to be difficult in conventional known delivery systems. In the context of the present invention, reference to cyclosporine also encompasses any macrolide of the cyclosporines family (i.e. cyclosporine A, cyclosporine B, cyclosporine C, cyclosporine D, cyclosporine E, cyclosporine F, or cyclosporine G), as well as any of its pharmaceutical salts, derivatives or analogues.

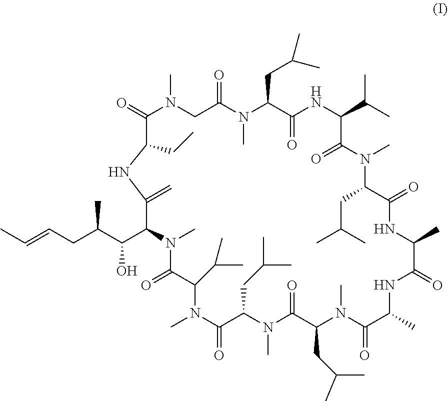 [SMILES: C=C1N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)C(C(C)C)C(=O)N(C)[C@H]1[C@H](O)[C@H](C)C/C=C/C]

According to some embodiments, the cyclosporine is cyclosporine A (CysA).

Both tacrolimus and pimecrolimus are utilized in dermatology for their topical anti-inflammatory properties in the treatment of atopic dermatitis. These non-steroidal medications down-regulate the immune system. Tacrolimus is manufactured as 0.03% and 0.1% ointment while pimecrolimus is distributed as a 1% cream; both are routinely applied twice daily to the affected area until clinical improvement is noted.

In some embodiments, the at least one non-hydrophilic agent is tacrolimus.

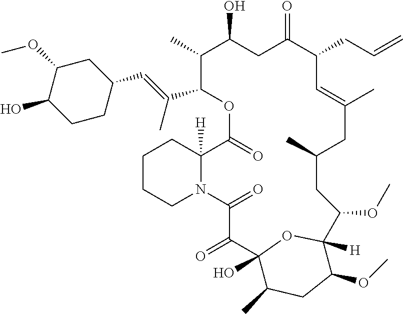 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In some embodiments, the at least one non-hydrophilic agent is pimecrolimus.

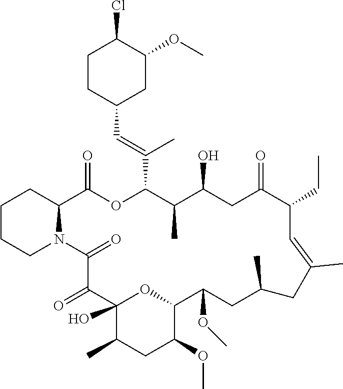 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](Cl)[C@H](OC)C3)[C@H](C)[C@@H](O)CC1=O)[C@H](C)C[C@@H]2OC]

In some embodiments, the nanoparticles comprise between about 0.1 and 10 wt % of the at least one non-hydrophilic material, e.g., cyclosporine.

The cannabis lipophilic extracted derivative used in accordance with the invention is an active, a composition or a combination thereof obtained from a cannabis plant by means known in the art. The extracted derivatives apply to purified as well as crude dry plant materials and extracts. There are number of methods for producing a concentrated cannabis-derived material, e.g., filtration, maceration, infusion, percolation, decoction in various solvents, Soxhlet extraction, microwave- and ultrasound-assisted extractions and other methods.

The cannabis lipophilic plant extract is a mixture of phyto-derived materials or compositions obtained from the cannabis plant, most often from Sativa, Indica, or Ruderalis species. It should be appreciated that the material composition and other properties of the extract may vary and further may be tailored to meet the desired properties of a combination therapy according to the invention.

As the cannabis plant extract is obtained by, e.g., extraction directly from a cannabis plant, it can include a combination of several naturally occurring compounds among them the lipophilic derivative, i.e., tetrahydrocannabinol (THC), cannabidiol (CBD), the two main naturally occurring cannabinoids, and further cannabinoids such as one or a combination of CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether) and others.

While THC and CBD are the main lipophilic derivatives, the other components of the extracted fractions are also within the scope of such lipophilic derivatives.

Tetrahydrocannabinol (THC) refers herein to a class of psychoactive cannabinoids characterized by high affinity to CB1 and CB2 receptors. THC having a molecular formula C₂₁H₃₀O₂, has an average mass of approximately 314.46 Da, and a structure shown below.

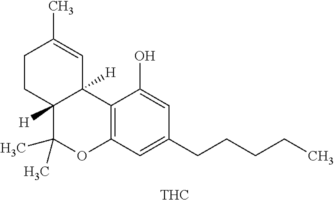

Cannabidiol (CBD) refers herein to a class of non-psychoactive cannabinoids with a low affinity to CB1 and CB2 receptors. CBD, having a formula C₂H₃₀O₂, has an average mass of approximately 314.46 Da, and a structure shown below.

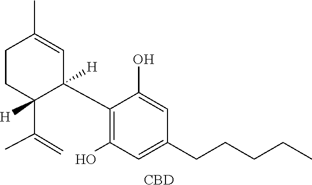

The terms ‘THC’ and ‘CBD’ herein further encompass isomers, derivatives, or precursors of these molecules, such as (−)-trans-Δ9-tetrahydrocannabinol (Δ9-THC), Δ8-THC, and Δ9-CBD, and further to THC and CBD derived from their respective 2-carboxylic acids (2-COOH), THC-A and CBD-A.

The “PLGA nanoparticles” are nanoparticles made of a copolymer of polylactic acid (PLA) and polyglycolic acid (PGA), the copolymer being, in some embodiments, selected amongst block copolymer, random copolymer and grafted copolymer. In some embodiments, the PLGA copolymer is a random copolymer. In some embodiments, the PLA monomer is present in the PLGA in excess amounts. In some embodiments, the molar ratio of PLA to PGA is selected amongst 95:5, 90:10, 85:15, 80:20, 75:25, 70:30, 65:35, 60:40, 55:45 and 50:50. In other embodiments, the PLA to PGA molar ratio is 50:50 (1:1).

The PLGA may be of any molecular weight. In some embodiments, the PLGA has an averaged molecular weight of at least 20 KDa. In some embodiments, the polymer has an averaged molecular weight of at least about 50 KDa. In some other embodiments, the polymer has an averaged molecular weight of between about 20 KDa and 1,000 KDa, between about 20 KDa and 750 KDa, or between about 20 KDa and 500 KDa.

In some embodiments, the polymer has an averaged molecular weight different from 20 KDa.

In some embodiments, the PLGA optionally has an averaged molecular weight of at least about 50 KDa or an averaged molecular weight selected to be different from an averaged molecular weight between 2 and 20 KDa.

Depending on the desired rate and/or mode of release, as well as the administration route of the at least one non-hydrophilic material from the nanoparticle, it may be contained (encapsulated) in the nanoparticle, embedded in the polymer matrix making up the nanoparticle and/or chemically or physically associated with the surface (whole surface or a portion thereof) of the nanoparticle. For some applications, the nanoparticle may be in the form of core/shell (termed hereinafter also as nanocapsule or NCs), having a polymeric shell and an oily core, the at least one non-hydrophilic active being solubilized within the oily core. Alternatively, the nanoparticles are of a substantially uniform composition, not featuring a distinct core/shell structure, into which the non-hydrophilic material is embedded; in such nanoparticles, that will be referred to herein as nanospheres (NSs), the material may be embedded within the polymer matrix, e.g., homogenously, resulting in a nanoparticle in which the concentration of material within the nanoparticle is substantially uniform throughout the nanoparticle volume or mass. In nanospheres an oil component may not be needed.

In some embodiments, the nanoparticle is in a form of nanosphere or a nanocapsule. In some embodiments, the nanoparticle is in the form of a nanosphere that comprises a matrix made of the PLGA polymer, and the non-hydrophilic material is embedded within the matrix.

In some embodiments, the nanoparticle is in the form of a nanocapsule that comprises a shell made of the PLGA polymer, the shell encapsulating an oil (or a combination of oils or an oily formulation) that solubilizes the non-hydrophilic material. The oil may be constituted by any oily organic solvent or medium (single material or mixture). In such embodiments, the oil may comprise at least one of oleic acid, castor oil, octanoic acid, glyceryl tributyrate and medium or long chain triglycerides.

In some embodiments, the oil formulation comprises castor oil. In other embodiments, the oil formulation comprises oleic acid.

The oil may be in the form of an oil formulation that may further comprise various additives, for example at least one surfactant. The surfactant may be selected from oleoyl macrogol-6 glycerides (Labrafil M 1944 CS), Polysorbate 80 (Tween® 80), Macrogol 15 hydroxystearate (Solutol HS15), 2-Hydroxypropyl)-β-cyclodextrin (Kleptose® HP), phospholipids (e.g. lipoid 80, phospholipon, etc.), tyloxapol, poloxamers, and any mixtures thereof.

In some embodiments, and as explained hereinabove, at least one cryoprotectant may be used to protect the nanoparticles integrity during lyophilization. Non-limiting examples of cryoprotectants include PVA and cyclodextrins such as 2-hydroxypropyl-β-cyclodextrin (Kleptose® HP) and others as recited herein.

The non-hydrophilic material, being a drug or an active agent, as recited herein, may be associated with the surface of said nanoparticle, e.g. by direct binding (chemical or physical), by adsorption onto the surface, or via a linker moiety, regardless of the type of nanoparticle used (for both NSs and NCs). Alternatively, when the nanoparticle is a nanosphere, the active agent may be embedded within the nanoparticle. When the nanoparticle is in the form of a nanocapsule, the active agent may be contained within a core of the nanoparticle.

In some embodiments, in the case where non-hydrophilic material is solubilized within an oil contained within the nanoparticle, e.g., in a core of a nanocapsule, the non-hydrophilic material may be solubilized within the core, embedded within the polymeric shell, or associated with the surface of the nanocapsule. When the nanoparticle is a nanosphere, the non-hydrophilic material may be embedded within the polymer.

In some embodiments, the nanoparticle may be associated with at least two different non-hydrophilic materials, each being associated to the nanoparticle in the same manner or different manners. When a plurality of active agents, e.g., at least two non-hydrophilic materials, the agents may be all non-hydrophilic materials or at least one of them may be a non-hydrophilic material. A combination of non-hydrophilic materials allows targeting of multiple biological targets or increasing affinity for a particular target.

The additional active agent to be presented with at least one non-hydrophilic material, may be selected from a vitamin, a protein, an anti-oxidant, a peptide, a polypeptide, a lipid, a carbohydrate, a hormone, an antibody, a monoclonal antibody, a therapeutic agent, an antibiotic agent, a vaccine, a prophylactic agent, a diagnostic agent, a contrasting agent, a nucleic acid, a nutraceutical agent, a small molecule of a molecular weight of less than about 1,000 Da or less than about 500 Da, an electrolyte, a drug, an immunological agent, a macromolecule, a biomacromolecule, an analgesic or anti-inflammatory agent; an enthelmintic agent; an anti-arrhythmic agent; an anti-bacterial agent; an anti-coagulant; an anti-depressant; an antidiabetic; an anti-epileptic; an anti-fungal agent; an anti-gout agent; an anti-hypertensive agent; an anti-malarial agent; an anti-migraine agent; an anti-, muscarinic agent; an anti-neuroplastic agent or immunosuppressant; an anti-protazoal agent; an anti-thyroid agent; an alixiolytic, sedative, hypnotic or neuroleptic agent; a beta-blocker; a cardiac inotropic agent; a corticosteroid; a diuretic agent; an anti-Parkinsonian agent; a gastro-intestinal agent; an histamine H1-receptor antagonist; a lipid regulating agent; a nitrate or anti-anginal agent; a nutritional agent; an HIV protease inhibitor; an opioid analgesic; capsaicin a sex hormone; a cytotoxic agent; and a stimulant agent, and any combination of the aforementioned.

Further, the nanoparticle may be associated with at least one non-active agent. While, in most general terms, the non-active agent has no direct therapeutic effect, it may modify one or more property of the nanoparticles. In some embodiments, the non-active agent may be selected to modulate at least one characteristic of the nanoparticle, such as one or more of size, polarity, hydrophobicity/hydrophilicity, electrical charge, reactivity, chemical stability, clearance and targeting and others. The non-active agent may, inter alia, improve penetrability of the nanoparticle, improve disperseability of the nanoparticles in liquid suspensions, stabilize the nanoparticle during lyophilization and/or reconstitution, etc. In some embodiments, the at least one non-active agent is capable of inducing, enhancing, arresting or diminishing at least one non-therapeutic and/or non-systemic effect.

As stated herein, the invention provides a lyophilized flaky dispersible dry powder comprising a plurality of the PLGA nanoparticles and non-hydrophilic material(s). The powder is a solid material, which may be in particulate form, that is dry of water. The term “dry” as used herein refers to any one of the alternatives: dry of water, free of water, absent of water, substantially dry (comprising no more than 1%-5% water), comprising only water of hydration, not being a water or an aqueous solution. In some embodiments, the amount of water does not exceed 7% wt. The powder may be anhydrous, namely having a water content of less than 3% by weight, or less than 2% by weight, or less than 1% by weight, relative to the total weight of the powder, and/or a composition which does not contain any added water, i.e. the water that may be present in the powder is more particularly bound water, such as water of crystallization of salts, or traces of water absorbed by the starting materials used in the production of the powder.

As known in the art, lyophilization refers to freeze-drying of a formulation by freezing it and then reducing the surrounding pressure to allow the frozen formulation to volatilize, evaporate or sublimate directly from the solid phase to the gas phase, leaving behind a dry powder, as defined. Thus, the dry lyophilized powder of the invention is a powder that has been obtained dry. In some embodiments, the powder may be obtained at the same degree of dryness by other methods, not by lyophilization for example by nanospraying (e.g., utilizing a nanospray dryer B-90 of Buchi, Flawill, Switzerland). Thus, the invention also provides a dry powder, not obtained by lyophilization.

The dry powder of the invention is provided as ready-for-reconstitution, in a form that may be re-dispersed by adding the powder into a pharmaceutically acceptable reconstitution liquid medium or carrier. The uniqueness of the powder of the invention resides in its stability to decomposition by way of separation of the active ingredients from the nanoparticle carriers, and also in the ability to tailor various reconstituted liquid formulations that are stable and may be administered and used in a variety of fashions. Examples of reconstitution mediums include water, water for injection, bacteriostatic water for injection, sodium chloride solutions (e.g., 0.9 percent (w/v) NaCl), glucose solutions (e.g., 5 percent glucose), a liquid surfactant, a pH-buffered solution (e.g., phosphate-buffered solutions), silicone-based solutions and others.

According to some embodiments, the reconstitution medium is an anhydrous silicone-based carrier that is free of water or is dry from water, as described herein, and as such holds the nanoparticles intact for long periods of time. The silicone-based carrier does not permit release of the nanoparticles' cargo until such a time when the nanoparticles come in contact with water, at which point the nanoparticles' cargo begins to discharge. This discharge may occur following application of the silicon-based formulation onto the skin and penetration of the nanoparticles into skin layers.

The silicone-based carrier is a liquid, viscous-liquid or semi-solid carrier, typically a polymer, oligomer or monomer that comprises siliconic building blocks. In some embodiments, the silicone-based carrier is at least one silicone polymer or at least one formulation of silicone polymers, oligomers and/or monomers. In some embodiments, the silicone-based carrier comprises cyclopentaxiloane, cyclohexasiloxane (such as ST-Cyclomethicone 56-USP-NF), polydimethylsiloxane (such as Q7-9120 Silicone 350 cst (polydimethylsiloxane)-USP-NF Elastomer 10), and others.

In some embodiments, the silicone-based carrier comprises cyclopentasiloxane and dimethicone crosspolymer. In some embodiments, the silicone-based carrier comprises cyclopentaxiloane and cyclohexasiloxane.

In some embodiments, the ready-for-reconstitution solid may be mixed in a semi-solid silicone elastomer blend comprising cyclohexasiloxane, cyclopentasiloxane, and polydimethylsiloxane polymer at weight ratios 80:15:3 respectively, w/w. In some embodiments, 2% of lyophilized nanoparticles comprising at least one non-hydrophilic material are dispersed in a formulation comprising cyclohexasiloxane, cyclopentasiloxane, and polydimethylsiloxane polymer at weight ratios 80:15:3 respectively, w/w, resulting in an active final concentration of 0.1%, w/w.

In some embodiments, such a formulation comprises further at least one preservative such as benzoic acid and/or benzalkonium chloride.

In some embodiments, the reconstitution medium is water-based.

For formulations intended for immediate use or use within a short period of time, e.g., of between 7 and 28 days, depending on the active ingredient, as recommended, for example, for water-sensitive active ingredients such as tacrolimus and antibiotics, the formulation may be formed in an aqueous or water-based medium comprising a powder of the invention and at least one water-based carrier, as defined. For example, such formulations may be ocular formulations, e.g., eye drops, or formulations for injection. Where the formulations are intended for prolonged use or storage as a ready-for-use formulation, then, the powder may be reconstituted in an anhydrous silicon-based liquid carrier.

The stability of formulations of the invention depends, inter alia, on the constitution of the formulation, the specific active ingredient(s) used, the medium in which the powder is reconstituted and storage conditions. Without wishing to be bound by theory, generally speaking, the stability of the formulations may be viewed and tested from two different directions:

1/stability relating to the active ingredient(s) contained within the lyophilized flaky powder, over time, as indicated in the data provided hereinbelow, for e.g., cyclosporine within an oily core. As demonstrated, such formulations are stable in castor oil core NCs, but not stable in oleic acid core NCs (Table 5 and Table 8). Stability tests over time, at 37° C., over 6 months, indicate that leakage and active content deviated from the initial values where the oil was oleic acid, whereas in castor oil the active was stable chemically and demonstrated no increase in leakage. That means that these lyophilized powders can normally be stored at room temperature for at least about 3 years.

2/stability is NCs dispersed in a topical formulation. Under the test conditions, over 6 months at the three different temperatures, only with Castor oil in NCs the active e.g., CsA, was maintained stable and did not leak more than 10% towards the external phase of the topical formulation.

Thus, the invention further provides a dermatological (topical) formulation comprising a plurality of NC nanoparticles, each comprising at least one non-hydrophilic material in an oily core, the core comprising castor oil.

Where ocular or injectable formulations are concerned, the dry flaky NCs behave similarly to NCs formulated for topical application (Table 10 and 17 below). Where a dispersed formulation is concerned for ocular formulations, dispersion of dry NCs of tacrolimus a sterile aqueous formulation, stability is maintained over a period of between 7 and 28 days, depending on the active ingredient and its sensitivity to the water.

For example, for a lyophilate reconstitution, NCs reconstitution stability in 1.45% glycerin solution (60 mg of lyophilized NCs were re-suspended in 350 uL of 1.45% glycerin in water to obtain isotonic formulation. Stability was evaluated at room temperature):

| | | After | After | After |
|---|---|---|---|---|
| | Initially | 7 days | 14 days | 21 days |
| Size(nm) | 171.6 | 177.7 | 179.5 | 168.5 |
| PDI | 0.13 | 0.127 | 0.118 | 0.153 |
| Tac | 0.57 | 0.57 | 0.56 | 0.51 |
| Content | | | | |
| (%) | | | | |
| Remarks | No | No | No | Aggregates |
| | aggregates | aggregates | aggregates | |

NCs reconstitution stability in 2.5% dextrose solution (60 mg of lyophilized NCs were re-suspended in 350 uL of 2.5% dextrose in water to obtain isotonic formulation. Stability was evaluated at room temperature):

| | | After | After | After |
|---|---|---|---|---|
| | Initially | 7 days | 14 days | 21 days |
| Size(nm) | 171.6 | 181 | 180.9 | 169.9 |
| PDI | 0.13 | 0.117 | 0.123 | 0.163 |
| Content | 0.57 | 0.57 | 0.55 | 0.50 |
| (%) | | | | |
| Remarks | No | No | No | Aggregates |
| | aggregates | aggregates | aggregates | |

As may be noted from the above results, the active, e.g., Tacrolimus, remained stable in this aqueous formulation at least 2 weeks at room temperature

Thus, the invention further provides a stable aqueous formulation comprising a powder of the invention for use over a period of between 7 and 28 days from the time of the formulation reconstitution. The invention further provides a stable anhydrous formulation, e.g., of at least two weeks, as shown above.

The choice of a carrier will be determined in part by the compatibility with the active agent (when used), as well as by the particular method used to administer the composition. Accordingly, a pharmaceutical composition (or a formulation) obtained following reconstitution of a powder in a liquid carrier may be formulated for oral, enteral, buccal, nasal, topical, transepithelial, rectal, vaginal, aerosol, transmucosal, epidermal, transdermal, dermal, ophthalmic, pulmonary, subcutaneous, intradermal and/or parenteral administrations.

In some embodiments, the formulations are configured or adapted for topical use. As known, human skin is made of numerous layers which may be divided into three main group layers: Stratum corneum which is located on the outer surface of the skin, the epidermis and the dermis. While the Stratum corneum is a keratin-filled layer of cells in an extracellular lipid-rich matrix, which in fact is the main barrier to drug delivery into skin, the epidermis and the dermis layers are viable tissues. The epidermis is free from blood vessels, but the dermis contains capillary loops that can channel therapeutics for transepithelial systemic distribution. While transdermal delivery of drugs seems to be the route of choice, only a limited number of drugs can be administered through this route. The inability to transdermally deliver a greater variety of drugs depends mostly on the requirement for low molecular weight (drugs of molecular weights not higher than 500 Da), lipophilicity and small doses of the drug.

The nanoparticles of this invention clearly overcome these obstacles. As noted above, the nanoparticles are able of holding an active ingredient such as cyclosporine and other active agents of a great variety of molecular weights and hydrophilicities. The delivery system of the invention permits the transport of the at least one non-hydrophilic agent across at least one of the skin layers, across the Stratum corneum, the epidermis and the dermis layers. Without wishing to be bound by theory, the ability of the delivery system to transport the therapeutic across the Stratum corneum depends on a series of events that include diffusion of the intact system or the dissociated therapeutic agent and/or the dissociated nanoparticles through a hydrated keratin layer and into the deeper skin layers.

The topical formulation may be in a form selected from a cream, an ointment, an anhydrous emulsion, an anhydrous liquid, an anhydrous gel, a powder, flakes or granules. The compositions may be formulated for topical, transepithelial, epidermal, transdermal, and/or dermal administration routes.

In some embodiments, a formulation is adapted for transdermal administration of at least one non-hydrophilic agent. In such embodiments, the formulation may be formulated for topical delivery of the non-hydrophilic agent across skin layers, and specifically across the Stratum Corneum. Where systemic effects of the non-hydrophilic agent are desired, the transdermal administration may be configured for delivery of the agent into the circulatory system of a subject.

Increasing stability of the nanoparticles in a formulation of the invention, e.g., for topical applications, may be achieved by formulating a carrier composition which is essentially or completely free of water. Thus, a topical composition which is free of water, or anhydrous, may be designed in a silicon-based carrier.

Similarly, a formulation composition may be configured for ophthalmic administration of the at least one non-hydrophilic agent. In some embodiments, the ophthalmic formulation may be configured for injection or eye drops.

In formulations designed for oral administration, administration by injection, administration by drip, administration in the form of drops, or any other form of administration which requires the formation of a suspension of nanoparticles, the solution can be comprised of, but not limited to, saline, water or a pharmaceutically acceptable organic medium.

The amount or concentration of nanoparticles, and the corresponding amount or concentration of the at least one non-hydrophilic agent in the nanoparticles, or overall in a formulation of the invention may be selected so that the amount is sufficient to deliver a desired effective amount of the non-hydrophilic agent to the target organ or tissue in the subject. The “effective amount” of the at least one non-hydrophilic agent may be determined by such considerations as known in the art, not only so that the amount of the agent is effective to achieve a desired therapeutic effect, but also to achieve a stable delivery system, as defined. Thus, depending, inter alia, on the particular agent used, the particular carrier system employed, the type and severity of the disease to be treated and the treatment regime, each formulation may be tailored to contain a predetermined amount that is effective not only at the time of formulation but more importantly at the time of administration. The effective amount is typically determined in appropriately designed clinical trials (dose range studies) and the person versed in the art will know how to properly conduct such trials in order to determine the effective amount. As generally known, the effective amount depends on a variety of factors including the affinity of the ligand to the receptor, its distribution profile within the body, a variety of pharmacological parameters such as half-life in the body, on undesired side effects, if any, on factors such as age and gender, and others.

The pharmaceutical formulations may comprise varying nanoparticle types or sizes, of different or same dispersion properties, utilizing different or same dispersing materials so that they facilitate one or more of targeted drug delivery and controlled release modalities, enhancement of drug bioavailability at the site of action (also due to a decreased clearance), reduction of dosing frequency, and minimization of side effects. The formulations and nanoparticles acting as delivery systems are capable of delivering the desired non-hydrophilic actives at a rate allowing their controlled release over at least about 12 hours, or in some embodiments, at least about 24 hours, at least about 48 hours, or in other embodiments, over a period of a few days. As such, the delivery system may be used for a variety of applications, such as, without limitation, drug delivery, gene therapy, medical diagnosis, and for medical therapeutics for, e.g., skin pathologies, cancer, pathogen-borne diseases, hormone-related diseases, reaction-by-products associated with organ transplants, and other abnormal cell or tissue growth.

The invention further provides a method of obtaining lyophilized dry powder, the powder comprising a plurality of PLGA nanoparticles, each nanoparticle comprising at least one non-hydrophilic material (drug), the method comprising lyophilizing a suspension of the PLGA nanoparticles to provide a dry lyophilized powder.

In some embodiments, the method comprises:
obtaining a suspension of PLGA nanoparticles comprising at least one hydrophobic material (drug); and
lyophilizing said suspension to provide a dry lyophilized flaky powder.

In some embodiments, the PLGA nanoparticles comprising the at least one non-hydrophilic material are obtained by forming an organic phase by dissolving PLGA in at least one solvent (such as acetone) containing at least one surfactant, at least one oil and at least one non-hydrophilic material (such as cyclosporine); introducing the organic phase into an aqueous phase (an organic medium or formulation), to thereby obtain a suspension comprising said nano carriers.

In some embodiments, the suspension is concentrated, e.g., by evaporation, and subsequently treated with at least one cryoprotectant (such as diluted with 10% HPβCD solution, at a volume ratio of 1:1) and lyophilized.

The so-lyophilized solid has a water content not exceeding 5% and may be further used as a ready-for-reconstitution powder.

The invention further provides a kit or a commercial package comprising a dry lyophilized powder and at least one liquid carrier; and instructions of use. In some embodiments, the liquid carrier is water or an aqueous solution or an anhydrous (water free) liquid carrier, as recited herein.

As demonstrated herein, formulations according to the invention may be generically used with different non-hydrophilic drug entities. Depending on the non-hydrophilic drug used, the formulation may be used in methods of treatment or prevention of different diseases and conditions. In some embodiments, the pharmaceutical formulations may be used to treat a condition or disorder typically treatable with one or more of the non-hydrophilic materials specifically recited herein. In some embodiments, said disease or condition is selected from graft-versus-host disease, ulcerative colitis, rheumatoid arthritis, psoriasis, nummular keratitis, dry eye symptoms, posterior uveitis, intermediate uveitis, atopic dermatitis, Kimura disease, pyoderma gangrenosum, autoimmune urticaria, and systemic mastocytosis.

The nanoparticles and pharmaceutical formulations of the present disclosure may be particularly advantageous to those tissues protected by physical barriers. Such barriers may be the skin, a blood barrier (e.g., blood-thymus, blood-brain, blood-air, blood-testis, etc), organ external membrane and others. Where the barrier is the skin, the skin pathologies which may be treated by the pharmaceutical formulations as described herein (at time when cyclosporine is combined with other actives) include, but are not limited to antifungal disorders or diseases, acne, psoriasis, atopic dermatitis, vitiligo, a keloid, a burn, a scar, xerosis, ichthoyosis, keratosis, keratoderma, dermatitis, pruritis, eczema, pain, skin cancer, and a callus.

The pharmaceutical formulations of the invention may be used to prevent or treat dermatologic conditions. In some embodiments, the dermatological conditions may be selected amongst dermatologic diseases, such as dermatitis, eczema, contact dermatitis, allergic contact dermatitis, irritant contact dermatitis, atopic dermatitis, infantile eczema, Besnier's prurigo, allergic dermatitis, flexural eczema, disseminated neurodermatitis, seborrheic (or seborrhoeic) dermatitis, infantile seborrheic dermatitis, adult seborrheic dermatitis, psoriasis, neurodermatitis, scabies, systemic dermatitis, dermatitis herpetiformis, perioral dermatitis, discoid eczema, Nummular dermatitis, Housewives' eczema, Pompholyx dyshidrosis, Recalcitrant pustular eruptions of the palms and soles, Barber's or pustular psoriasis, Generalized Exfoliative Dermatitis, Stasis Dermatitis, varicose eczema, Dyshidrotic eczema, Lichen Simplex Chronicus (Localized Scratch Dermatitis; Neurodermatitis), Lichen Planus, Fungal infection, Candida intertrigo, tinea capitis, white spot, panau, ringworm, athlete's foot, moniliasis, candidiasis; dermatophyte infection, vesicular dermatitis, chronic dermatitis, spongiotic dermatitis, dermatitis venata, Vidal's lichen, asteatosis eczema dermatitis, autosensitization eczema, skin cancers (non-melanoma), fungal and microbial resistant skin infections, skin pain or a combination thereof.

In further embodiments, formulations of the invention may be used to prevent or treat pimples, acne vulgaris, birthmarks, freckles, tattoos, scars, burns, sun burns, wrinkles, frown lines, crow's feet, café-au-lait spots, benign skin tumors, which in one embodiment, is Seborrhoeic keratosis, Dermatosis papulosa nigra, Skin Tags, Sebaceous hyperplasia, Syringomas, Xanthelasma, or a combination thereof; benign skin growths, viral warts, diaper candidiasis, folliculitis, furuncles, boils, carbuncles, fungal infections of the skin, guttate hypomelanosis, hair loss, impetigo, melasma, molluscum contagiosum, rosacea, scapies, shingles, erysipelas, erythrasma, herpes zoster, varicella-zoster virus, chicken pox, skin cancers (such as squamous cell carcinoma, basal cell carcinoma, malignant melanoma), premalignant growths (such as congenital moles, actinic keratosis), urticaria, hives, vitiligo, Ichthyosis, Acanthosis Nigricans, Bullous Pemphigoid, Corns and Calluses, Dandruff, Dry Skin, Erythema Nodosum, Graves' Dermopathy, Henoch-Schönlein Purpura, Keratosis Pilaris, Lichen Nitidus, Lichen Planus, Lichen Sclerosus, Mastocytosis, Molluscum Contagiosum, Pityriasis Rosea, Pityriasis Rubra Pilaris, PLEVA, or Mucha-Habermann Disease, Epidermolysis Bullosa, Seborrheic Keratoses, Stevens-Johnson Syndrome, Pemphigus, or a combination thereof.

In additional embodiments, the formulations may be used to prevent or treat dermatologic conditions that are associated with the eye area, such as syringoma, xanthelasma, Impetigo, atopic dermatitis, contact dermatitis, or a combination thereof the scalp, fingernails, such as infection by bacteria, fungi, yeast and virus, Paronychia, or psoriasis; mouth area, such as oral lichen planus, cold sores (herpetic gingivostomatitis), oral leukoplakia, oral candidiasis, or a combination thereof or a combination thereof.

According to some embodiments, the pharmaceutical composition may be used for treating or ameliorating at least one symptom associated with alopecia.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-E provide characterization of CsA loaded NCs. (A) XRD patterns of crystalized CsA (i), lyophilized CsA NCs (ii) and lyophilized blank NCs (iii). Transmission electron microscopy images of CsA-loaded PLGA NCs (B-C, Bar=100 nm). Cryo-SEM depictions of the lyophilized CsA-loaded NCs (D, D(i)) and the cryo-protective agent (E) incorporated in anhydrous silicone base following freeze fracturing. Scale bars=1 μm (D), 200 nm (D(i)), 2 μm (E).

FIGS. 2A-C present cutaneous biodistribution of CsA NCs. [³H]-CsA distribution in skin compartments determined by penetration assay in Franz cells. (A) SC upper layers, (B) lower SC and epidermis and (C) dermis, 6 and 24 hours following incubation of various oil compositions CsA-loaded NCs and the respective oil controls. Values are mean±SD. N=5. OL and LA mean oleic acid and Labrafil respectively.

FIGS. 3A-D show [3H]-CsA distribution in skin compartments determined by penetration assay in Franz cells. (A) SC upper layers, (B) lower SC and epidermis, (C) dermis and (D) receptor compartment, 6 and 24 hours following incubation of various oil compositions CsA-loaded NCs and the respective oil controls. Values are mean±SD. N=3.

FIG. 4 depicts the effect of different CsA formulations on contact hypersensitivity (CHS) in mice. Single treatment (20 μg/cm²) was topically applied to the mice shaved abdomen prior to challenge with 1% Oxazolone. Ear response elicitation was performed five days later on the right ear lobe (0.5% Oxazolone) and the ear swelling was presented by the differences between the right and left ears. Values are mean±SE. N=5. *P<0.05.

FIG. 5 shows NEs' droplets size distribution obtained by MasterSizer.

FIGS. 6A-C provide Cryo-TEM pictures of (A) NE-6, (B) NE-7, (C) NE-8.

FIGS. 7A-B provide Tacrolimus amount retained in the cornea/area unit (A) and Tacrolimus concentration in the receptor fluid (B) 24 h following incubation of NEs and the oil control. Values are mean±SD based on three replicates. *P<0.05 between the NEs and the oil control.

FIGS. 8A-B are TEM pictures of Tacrolimus loaded Nanocapsules (A) before and (B) after lyophilization following aqueous reconstitution.

FIGS. 9A-B depict Tacrolimus amount retained in the cornea/area unit (A) and Tacrolimus concentration in the receptor fluid (B) 24 h following incubation of NCs and the oil control. Values are mean±SD based on six replicates. *P<0.05, **P<0.01 between the NEs and the oil control in (A) and between the indicated treatments in (B).

FIG. 10 provides Tacrolimus concentration in the receptor fluid 24 h following incubation of NC-2 lyophilized and NEs. Values are mean±SD based on three replicates. *P<0.05, **P<0.01 between the NEs and lyophilized NC-2.

FIG. 11 provides MTT viability assay performed 72 h post treatment application on incubated ex vivo pig corneas. Control represents untreated corneas, negative control is Labrasol-treated corneas. Values are mean±SD based on three replicates.

FIG. 12 shows Epithelial thickness measurement on histological ex vivo pig corneas incubated during 72 h. Values are mean±SD based on three replicates.

### DETAILED DESCRIPTION OF EMBODIMENTS

### I. Experimental

### 1) Active and Excipients Included in the Topical Preparation

| Materials | Company |
|---|---|
| Cyclosporine A (CsA) USP | Teva Czech industries S.R.O. |
| | (Opava-Komarov, Czech |
| | Republic) |
| PLGA 100 kDa (Poly-D,L-lactide-co- | Lactel (Durect corporation, |
| glycolide at 50:50 blend of LA:GA) | Birmingham, USA) |
| not listed but marketed in | |
| Trelstar* (US product) | |
| Oleic acid USP or castor oil USP | Fisher chemical, USA or |
| | Lamotte, France, respectively |
| Labrafil M 1944 CS (Oleoyl | Gatefosse (Saint Priest cedex, |
| macrogol-6 glycerides EP), USP-NF | France) |
| Tween ® 80 (Polysorbate 80), USP | Ziv Chemical Ltd (Ashkelon, |
| | Israel) |
| Solutol ® HS 15 (Macrogol 15 | BASF (Ludwigshafen, |
| hydroxystearate), USP | Germany) |
| Kleptose ® HP | Roquette (Lestrem cedex, |
| ((2-Hydroxypropyl)-β-cyclodextrin), | France) |
| USP-NF | |
| ST-Cyclomethicone 56- USP-NF | Dow Corning (Seneffe, |
| (Cyclopentaxiloane and | Belgium) |
| Cyclohexasiloxane) | |
| Q7-9120 Silicone 350 cst | Dow Corning (Seneffe, |
| (Polydimethylsiloxane)-USP-NF | Belgium) |
| Elastomer 10 (Cyclopentasiloxane | Dow Corning (Seneffe, |
| and Dimethicone crosspolymer), DMF | Belgium) |

*TRELSTAR DEPOT is a sterile, lyophilized biodegradable microgranule formulation supplied as a single-dose vial containing triptorelin pamoate (3.75 mg as the peptide base), 170 mg poly-d,l-lactide-co-glycolide, 85 mg mannitol, USP, 30 mg carboxymethylcellulose sodium, USP, 2 mg polysorbate 80, NF. A monthly intramuscular injection following reconstitution.

### 2) Preparation of Blank and Drug-Loaded NCs

The various PLGA nanocarriers were prepared according to the well-established solvent displacement method (Fessi et al., 1989). Briefly, the polymer poly lactic-co-glycolic acid (PLGA) 100K (50:50 blend of lactic:glycolic acid), was dissolved in acetone containing 0.2% w/v Tween® 80 and up to 1% w/v blend of different oils at different compositions, at a concentration of 0.6% w/v. CsA was added at various concentrations into the organic phase, that was added to the aqueous phase containing 0.1% w/v Solutol® HS 15, resulting in the formation of NCs. The suspension was stirred at 900 rpm over 15 min and then concentrated by evaporating 80% of the initial aqueous medium by reduced pressure evaporation. The NCs dispersed in aqueous media were diluted with 10% HPβCD solution, at a volume ratio of 1:1, prior to lyophilization in epsilon 2-6 LSC Pilot Freeze Dryer (Martin Christ, Germany). Finally, semi-solid anhydrous preparations of blank and CsA NCs consisted of semi-solid silicone elastomer blend, cyclohexasiloxane (and) cyclopentasiloxane, polydimethylsiloxane polymer and lyophilized blank NC or CsA NCs at weight ratios 80:15:3:2 respectively. In fact, 2% of lyophilized CsA NCs were dispersed in the medicated formulation resulting in a final concentration of CsA of 0.1%, w/w in the final tested formulation.

In addition, benzoic acid and/or benzalkonium chloride may also be incorporated for preservation purposes.

### 3) Physicochemical Evaluation Protocols of CsA NCs Alone and in the Topical Formulation

Physicochemical Evaluation of the NCs Concentrated in Aqueous Suspension (PLGA Concentration:15 mg/mL)

3.1) Particle-Size and Zeta Potential Measurements

Mean diameter and zeta potential of the NCs were characterized using Malvern's Zetasizer (Nano ZSP) at 25° C. For the sample preparation, 10 μL of the concentrated dispersion was diluted into 990 μL HPLC water.

Sample

| | |
|---|---|
| Material | Polystyrene latex |
| Dispersant | Water |
| General options | Size: Mark-Houwink parameters (use |
| | dispersant viscosity as sample viscosity) |
| | Zeta: Model Smoluchowski (use |
| | dispersant viscosity as sample viscosity) |
| Temperature | 25° C. |
| | Equilibration time (s): 120 |
| Cell | Disposable folded capillary cells |
| | DTS1070 |

Measurement

Size: 173° C. Backscatter (NIBS default)
Measurement duration: Automatic
Number of measurements: 3
Delay between measurement (seconds): 0
Zeta: Measurement duration: Automatic
Minimum runs: 10
Maximum runs: 100
Number of measurements: 3
Delay between measurement (seconds): 0

3.2) CsA Loading Efficiency Determination

10 μL of the concentrated dispersion was diluted into 990 μL Acetonitrile (HPLC grade) and the CsA. The amount of CsA was quantified by HPLC as described later (factor dilution×100).

4) Physicochemical Evaluation of the Lyophilized NCs

4.1) Particle-Size and Zeta Potential Measurements

Mean diameter and zeta potential of the NCs were characterized using Malvern's Zetasizer (Nano ZSP) at 25° C. For the sample preparation, about 20 mg of the lyophilized NCs was dissolved in 1 mL HPLC water. Then 10 μL of the reconstituted lyophilized NCs was diluted into 990 μL HPLC.

4.2) Water Content Determination

The water content in the lyophilized NCs was determined by Karl Fischer method (KF) (Coulometer 831+KF Termoprep (oven) 860; Metrohm). The oven was set to 150° C. and the oven's airflow was set to 80 ml/min. The instrument was calibrated by oven standart (Hydranal-Water standard KF-oven, 140-160° C., Fluka, Sigma-aldrich) and triplicate blank was tested before each use in order to set the drift. For sample preparation approximately 20 mg of lyophilized NCs was weighted in a vial.

4.3) Acetone Content Determination

In order to determine traces of acetone in the lyophilized NCs, we utilized the dead space sampling of 90° C. pre-heated vial coupled to GCMS instrument.

4.4) CsA Content Determination

30 mg of the lyophilized NCs were dissolved in 1 mL HPLC water. Then, 10 μL of the reconstituted lyophilized NCs was added into 490 μL HPLC water. 500 μL Acetonitrile was also added. Finally, 250 uL of the prepared sample was diluted into 750 μL Acetonitrile (factor dilution×400). The amount of CsA was quantified by HPLC as described later.

4.5) Determination of Free CsA

Protocol validation: About 5 mg of CsA solution (28% w/w), dissolved in oleic acid:labrafil, were added to 30 mg of blank lyophilized NCs. CsA was completely extracted by Tributyrin as described below and 100% of CsA was recovered.

Free CsA in NCs lyophilized: Free CsA was evaluated by extracting the lyophilized NCs with Tributyrin. Approximately 15 mg of lyophilized NCs were weighted in a 4 mL vial and then 2.5 mL of Tributyrin were added. The solutions were vortexed for 30 s and further centrifuged (14 000 rpm, 10 min) (Mikro 200R, Hettich). Then, 100 μL of the supernatant was diluted in 1900 μL Acetonitrile, the solution was vortexed and then centrifuged (14 000 rpm, 10 min). Finally, 800 μL of the supernatant was collected and evaluated by HPLC (factor dilution×50). CsA levels represent the non-encapsulated CsA in the lyophilized NCs.

### 4) Anhydrous Topical Preparation

An anhydrous semi-solid base consisting of 80% Elastomer 10, 16% ST-Cyclomethicone 56-NF and 4% Q7-9120 Silicone 350 cst was prepared. Then, 2% lyophilized NCs was dispersed in the base. When small scales were prepared, the mixture was stirred using head stirrer set to 1800 rpm. For large scale preparation, up to 1 kg, IKA® LR 1000 basic reactor was used (100 rpm, at temperature controlled conditions).

### 5) Physicochemical Evaluation of the Anhydrous Semi-Solid Preparation

5.1) Particle-Size and Zeta Potential Measurements

Mean diameter and zeta potential of the NCs were characterized using Malvern's Zetasizer (Nano ZSP) at 25° C. For the sample preparation, 200 mg of the anhydrous semi-solid preparation were dissolved in 2 mL HPLC water. The sample was vortexed and further centrifuged (4 000 rpm, 10 min). Then, 1.2 mL of the supernatant was collected and centrifuged again (14 000 rpm, 10 min). Finally, 1 mL of the obtained supernatant was collected and evaluated.

5.2) CsA Content Determination (to be Modified)

200 mg of the anhydrous semi-solid preparation were dissolved in 2 mL DMSO in a 4 mL vial. The sample was shacked 30 min at 37° C. and then centrifuged (4 000 rpm, 10 min). 1 mL of the supernatant was centrifuge (14 000 rpm, 10 min). Finally, 10 μL of the supernatant was diluted into 990 μL Acetonitrile (factor dilution×200). The amount of CsA was quantified by HPLC as described later.

5.3) Determination of Free CsA

Protocol validation: About 1.5 mg of CsA solution (28% w/w), dissolved in oleic acid:labrafil, were added to added to 500 mg of a silicone base. CsA was extracted by Tributyrin as described below. At least 80% of CsA was recovered.

Free CsA in the anhydrous semi-solid preparation: The free CsA was evaluated using an extraction procedure. Approximately 500 mg of the anhydrous semi-solid preparation were weighted in a 4 mL vial and then 2.5 mL Tributyrin were added. The solution was vortexed and further centrifuged (14 000 rpm, 10 min). Then, 100 μL of the supernatant was diluted in 1900 μL Acetonitrile, then the solution was vortexed and centrifuged (14 000 rpm, 10 min). Finally, 800 μL of the supernatant was collected and evaluated by HPLC (factor dilution×50).

### 6) HPLC Method for CsA Quantification

10 μl of samples were injected into an HPLC system consisting of a pump, autosampler, column oven and UV detector (Dionex ultimate 300, Thermo Fisher Scientific). With 5 μm XTerra MS C8 column (3.9×150 mm) (Waters corporation, Mildfold, Mass., USA), identification of CsA was obtained at the wavelength of 215 nm. The column was thermostated at 60° C. CsA determination was achieved using a mobile phase consisted of a mixture of Acetonitrile:water (60:40 v/v) which elicited a retention time of 6.6 min. CsA stock solution (200 μg/mL) was prepared weighting 2 mg CsA in a 20 mL scintillation vial and adding 10 mL Acetonitrile. The stock was vortexed and calibration curve was prepared at concentration ranging from 1 to 100 μg/mL.

Calibration Curve Preparation

| Concentration | CsA stock | Acetonitrile |
|---|---|---|
| (μg/mL) | (μL) | (μL) |
| 0 | 0 | 1000 |
| 1 | 5 | 995 |
| 2.4 | 12 | 988 |
| 5 | 25 | 975 |
| 10 | 50 | 950 |
| 20 | 100 | 900 |
| 25 | 125 | 875 |
| 50 | 250 | 750 |
| 100 | 500 | 500 |

Calibration Curve

CsA content in the lyophilized powder was determined as described in equation (1).

$$\% CsA ( w \text{/} w ) = \frac{Drug amount}{Lyophilized powder amount} . \qquad ( 1 )$$

### 7) Morphological Evaluation

Finally, two techniques were used for morphological evaluation: Transmission Electron Microscope (TEM) and Cryo-Scanning Electron Microscope (Cryo-SEM). Morphological evaluation was performed using transmission electron microscopy (TEM) (Philips Technai F20 100 KV) following negative staining with phosphotungstic acid and by cryo-scanning electron microscopy (Cryo-SEM), (Ultra 55 SEM, Zeiss, Germany). In the cryo-SEM method, the sample was sandwiched between two flat aluminum platelets with a 200 mesh TEM grid used as a spacer between them. The sample was then high-pressure frozen in a HPM010 high-pressure freezing machine (Bal-Tec, Liechtenstein). The frozen samples were mounted on a holder and transferred to a BAF 60 freeze fracture device (Bal-Tec) using a VCT 100 Vacuum Cryo Transfer device (Bal-Tec). After fracturing at a temperature of −120° C. samples were transferred to the SEM using a VCT 100 and were observed using secondary back-scattered and in-lens electrons detectors at 1 kV at a temperature of −120° C. X-ray diffraction (XRD) measurements were performed on the D8 Advance diffractometer (Bruker AXS, Karlsruhe, Germany) with a secondary Graphite monochromator, 2° Sollers slits and 0.2 mm receiving slit. XRD patterns within the range 2° to 55° 20 were recorded at room temperature using CuKα radiation (λ=1.5418 A) with the following measurement conditions: tube voltage of 40 kV, tube current of 40 mA, step-scan mode with a step size of 0.02° 20 and counting time of 1 s/step. The calculations of degree of crystallinity were performed according to the method described by Wang et al (Wang et al., 2006). EVA 3.0 software (Bruker AXS) was used for all calculations. The equation for calculation of the degree of crystallinity is as follows: DC=100%·Ac/(Ac+Aa) where DC is the degree of crystallinity, Ac and Aa are the crystalline and amorphous areas on the X-ray diffractogram.

### 8) Porcine Tissue Treatment

Trimmed porcine ear skin, approximately 750 μm thick, was purchased from Lahav Animal Research Institute (Kibbutz Lahav, Israel), cleaned carefully and the dermatomed skin was either treated or stored frozen at −20° C. for up to a maximum of one month before use. Skin integrity was ensured by measuring transepidermal water loss (TEWL) (Heylings et al., 2001) using a VapoMeter device (Delfin Technologies, Finland). Only skin samples with TEWL values of ≤15 g h⁻¹ m² were used in the experiments (Weiss-Angeli et al., 2010).

### 9) Ex Vivo DBD Experiments

The excised pig skin was placed on Franz diffusion cells with the acceptor compartment containing 10% ethanol in PBS (pH 7.4). Various doses of radioactivity, equivalent to 937.5 μs of CsA, in NC formulations and respective controls were applied to the mounted skin. At different time intervals, the distribution of radioactively-labeled CsA was determined in several skin compartments. First, the remaining formulation on the skin surface was collected by serial washings and, combined with the first strip collected by D-SQUAME® skin sampling discs (CuDERM Corporation, Dallas, USA), made up the donor compartment. The subsequent 10 strips, consisting of five sequential tape stripping couples, were pooled as upper SC. Viable epidermis, containing also the lower SC, was heat-separated (1 min in PBS at 56° C.) from the dermis (Touitou et al., 1998). Then, the various separated layers were chemically dissolved with Solvable®. It should be emphasized that the remaining skin residuals were also digested in Solvable® and the residual radioactivity found was negligible. Aliquots of the receptor fluid were also collected. All the radioactive compounds were determined in Ultima-gold® scintillation liquid in a Tri-CARB 2900TR beta counter.

### III Results and Discussion

### 1) Preparation and Characterization of CsA-Loaded Various Nanocarriers

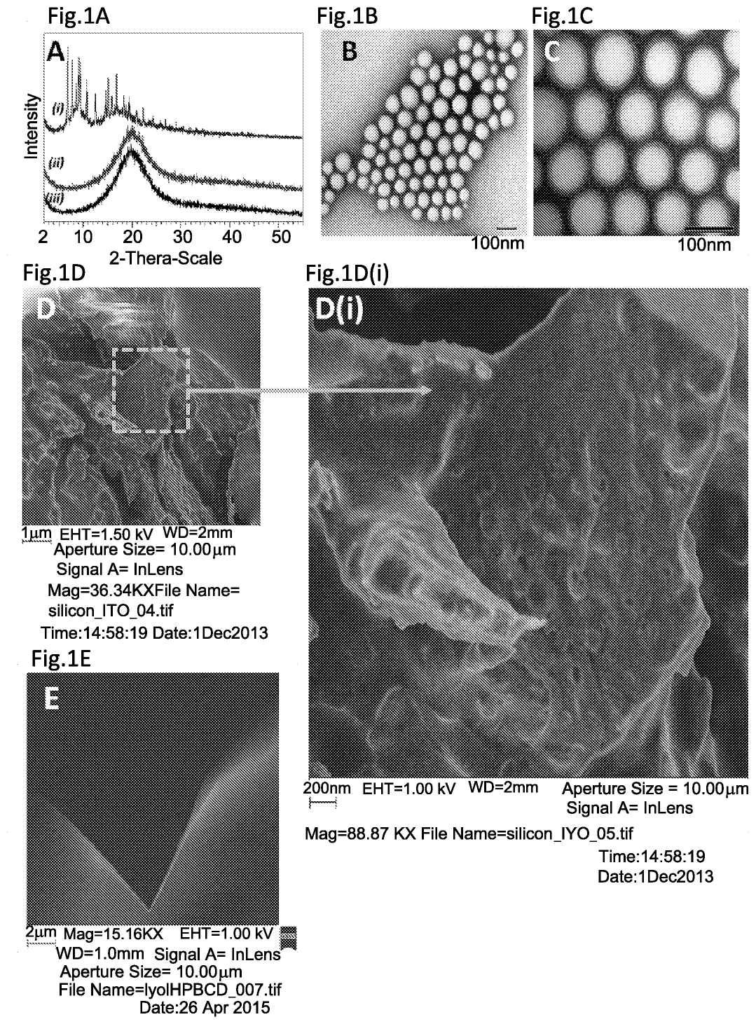

Various nanoparticulate formulations were prepared for this study, and their physical characteristics are summarized in Table 1. The mean diameter of the various nanocarriers varied from 100 to 200 nm with a relatively narrow distribution range as reflected by the low PDI values obtained. MCT-containing CsA NCs mean diameter was two-fold higher than that of the CsA NSs, while the variation of the oil core had a lesser effect on the particle size distribution of NCs (Table 1). The incorporation of the active agent CsA, with or without oil presence, did not alter the negatively-charged nature of the smooth and spherical PLGA-based NPs surfaces. High drug encapsulation efficiency (92.15% recovery) lead to the drug content of 4.65% (w/w) in the lyophilized powder only when the oil core in the NCs consisted of oleic acid:labrafil (Table 1). The main concern from the dispersion of drug loaded NCs in topical formulations is the leakage of the active cargo from the nanocarriers towards the external phase of the topical formulation, resulting in significant damage to the transport efficiency of the active through the skin. Furthermore, NCs of PLGA are water sensitive and may degrade slowly in aqueous formulations. Therefore, they need to be freeze-dried and incorporated within an appropriate water-free topical formulation. The NCs were efficiently dispersed in the silicone blend as confirmed by freeze-fracture cryo-SEM depictions [FIG. 1.D-D(i)]. According to the X-ray diffraction (XRD) patterns shown in FIG. 1A, it can be noted that the typical peaks of crystalline CsA (i), are missing from either blank (iii) or CsA-loaded NCs (ii) diffractions. This may imply that, when incorporated within NCs, the physical state of CsA is amorphous rather than crystalline. TEM images confirm the spherical shape and homogenous distribution of both blank and drug-loaded NCs in aqueous media (FIGS. 1B-C). As shown in FIG. 1D the lyophilized NCs form rough and uneven lattices in contrast to the smooth surface of HPßCD with no NCs (FIG. 1E). A closer look at the freeze fracture lyophilized NCs powder reveals spherical NCs embedded within cryoprotectant [FIG. 1D(i)]. The selection of the adequate formulation was based on two criteria, including the encapsulation efficiency and the resistance to the lyophilization stress. From the five formulations only the MCT and the oleic:labrafil containing CsA NCs succeeded to pass the lyophilization stress although it was more difficult to achieve a good lyophilized cake because of the higher oil concentration compared to oleic acid. Moreover, the oleic:labrafil formulation was selected because of the high encapsulation efficiency which contained 92.15% of the theoretical drug amount. This oil core combination was apparently the most efficient in retaining the CsA within the NCs during the formation process of the NCs before and after the lyophilization process (Table 1).

TABLE 1

| | | | | | | Encapsulation | Drug |
|---|---|---|---|---|---|---|---|
| | Oil | CsA^{a} | | | Zeta | efficiency | Content |
| Formulation | %, w/w | %, w/w | Mean | DI^{b} | potential | (%)^{c} | (%, w/w) |
| CsA loaded (oleic) NCs | 36 | 5.4 | 153.8 ± 1.8 | 0.15 | −40.6 | 70.62 | NAe |
| CsA-loaded (oleic:labrafil) NCs | 13 | 5 | 162.0 ± 0.75 | 0.06 | −36.0 | 92.15 | 4.65 |
| CsA-loaded (MCT) NCs | 36 | 5.4 | 192.8 ± 3.1 | 0.17 | −35.2 | 78.73 | 4.25 |
| CsA loaded (Tributyrin) NCs | 36 | 5.4 | 122.1 ± 2.7 | 0.17 | −35.6 | 69.54 | NAe |
| CsA-loaded NSs | 0 | 4.8 | 106.7 ± 0.2 | 0.08 | −34.5 | 54.0 | NAe |

Composition and properties of the different nanocarrier formulations^{:}
^{a}Initial drug concentration,
^{b}PDI = poly dispersity index,
^{c}prior lyophilization,
^{d}after lyophilization,
^{e}NA = data not available.

### 2) Cutaneous Biodistribution of CsA NCs Using Fresh Pig Skin in an Ex Vivo Model

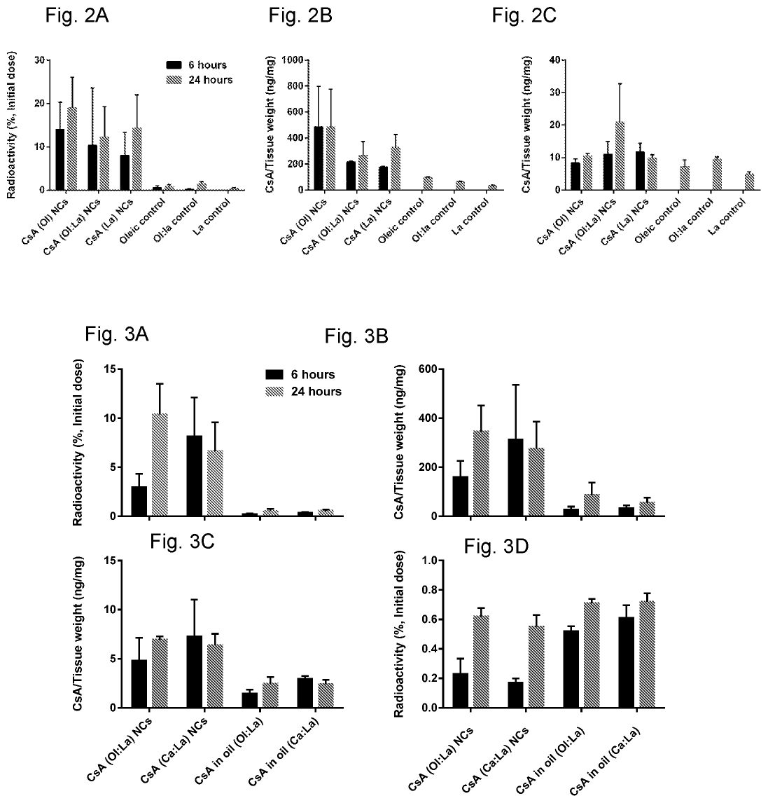

The results reported in FIG. 2 exhibit the ex vivo cutaneous distribution of CsA in the different skin compartments following topical application of various oil compositions-[³H]-CsA-loaded NCs and the respective oil controls at 6- and 24-hour incubation periods in Franz cells. [³H]-CsA distribution in the upper SC layers is depicted in FIG. 2A and consisted of the summation of five sequential tape stripping composed each of two separated consecutive tape stripping extractions (altogether 10 tape stripping's). Elevated levels of radioactive CsA, about 15% of the initial dose applied, were detected after 6 h in SC upper layers following topical application of the different CsA NC formulations. It should be noted that, when the respective oil controls were administered, low levels of [³H]-CsA, not exceeding 1.5% of the initial dose, were recorded in the SC (FIG. 2A). It was further found that in the viable epidermis layer of each skin sample, the calculated equivalent CsA concentrations (parent drug and probably some metabolites) from the loaded CsA NC formulations were significantly higher than respective oil formulations, as presented in FIG. 2B. Notably, CsA scarcely penetrated to the viable epidermis layers when administered in respective oil controls at any time point. In contrast, when CsA was encapsulated within NCs, higher concentrations of CsA were observed at 6 and 24 hours following application. Between 300 and 500 ng CsA per mg tissue weight were recovered at each time point. Although a similar pattern was observed in the dermis compartment (FIG. 2C), CsA concentration (10-20 ng/mg tissue weight) was much lower. It should be emphasized that no statistically significant differences between the various NC formulations, regardless of the oil core composition, were observed at any time point for all compartments investigated. On the other hand, in the receptor compartment fluids, the [³H]-CsA levels were less than 1% from the initial radioactivity at every time interval regardless of the treatment applied (data not shown).

When following lyophilization and reconstitution of the lyophilized powder into a NC aqueous dispersion, it was noted surprisingly that the amount of CsA leaked at time 0 was very significant with the oleic:labrafil oil core above 10% as shown also in Table 5 whereas surprisingly with castor oil:labrafil at the same ratio, the leakage was markedly less than 10% as noted again in Table 5.

Drug based nanoparticle (NP) formulations have gained considerable attention over the past decade for their use in various drug formulations. The major goals in designing polymeric NPs as a delivery system are to control particle size and polydispersity, maximize drug encapsulation efficiency and drug loading, and optimize surface properties and release of pharmacologically active agents to achieve a site-specific action of the drug at the therapeutically optimal desired rate and dose regimen.

To avoid any future problem, for the optimization process, our aim was to optimize the encapsulation CsA efficiency using selected oil compositions either oleic acid:labrafil or castor oil:labarafil ratio of 1:1 with PLGA (Lactel Ltd 100K E) or PLGA 17K of Purac Ltd. All the experimental conditions were identical except the nature of the oil (oleic acid versus castor oil).

The NPs formulation is based on CsA loaded poly-(lactic acid-co-glycolic acid) nanocapsules (PLGA-CsA).

The PLGA nanocapsules were prepared as follow: the polymer poly lactic-co-glycolic acid (PLGA) 100K (50:50 blend of lactic:glycolic acid), was dissolved in acetone containing 0.2% w/v Tween® 80 and 0.8% w/v blend of different oils at different compositions, at a concentration of 0.6% w/v. CsA was added at various concentrations into the organic phase, that was then added to the aqueous phase containing 0.1% w/v Solutol HS 15, resulting in the formation of nanocapsules (NCs). The suspension was stirred at 900 rpm over 15 min and then concentrated to 20% of the initial aqueous volume (assuming total removal of the acetone) by reduced pressure evaporation. The composition of the formulation is depicted in Table 2.

The NCs dispersed in aqueous media were diluted with a 10% HPβCD aqueous solution, at volume ratio of 1:1, prior to lyophilization in Epsilon 2-6 LSC Pilot Freeze Dryer (Martin Christ, Germany).

TABLE 2
List of ingredients and respective amounts for a typical lab
batch of 150 ml using castor oil:labrafil ratio of 1:1.

| Lab scale | Amount, mg |
|---|---|
| Organic phase | |
| Cyclosporine A | 150 |
| Castor oil | 200 |
| Labrafil | 200 |
| Tween 80 | 100 |
| PLGA (Lactel 100K E) | 300 |
| Acetone | 50 ml |
| Aqueous phase | |
| Solutol | 100 |
| Water | 100 ml |
| Total volume | 150 ml |

The lyophilization process of the 150 ml batches is described in Table 3

TABLE 3
description of the process parameters selected for the
Lyophilization of the lab batch (total time: ~17 hr)

| | | Time | Temp. | Vacuum |
|---|---|---|---|---|
| Sec. | Process phase | (h:min) | (° C.) | (mbar) |
| 1 | Loading | 00:00 | 20 | — |
| 2 | Freezing | 01:00 | −35 | — |
| 3 | Freezing | 01:00 | −35 | — |
| 4 | Sublimation | 00:15 | −35 | 1.03 |
| 5 | Sublimation | 00:15 | −20 | 1.03 |
| 6 | Sublimation | 00:10 | −10 | 0.94 |
| 7 | Sublimation | 04:00 | 0 | 0.94 |
| 8 | Sublimation | 05:00 | 20 | 0.94 |
| 9 | Second drying | 05:00 | 20 | 0.001 |
| | Total time | 16.40 | | |

It can be noted that with oleic acid:labrafil, the lyophilization process induced a stress which harmed the wall coating integrity of the NCs either using the 17K or 100K molecular weight PLGA (Table 5).

The different values for the various properties of the typical batch described in Table 2 and prepared with castor oil:labrafil are depicted in Table 4.

TABLE 4
Results of NCs formulation suspension and
lyophilized powder following reconstitution

| | | |
|---|---|---|
| NCs suspension | NCs diameter (nm) | 117.4 ± 12.9 |
| | PDI (nm) | 0.09 ± 0.01 |
| | CsA content (% w/w) | 14.3 ± 1.3 |
| | CsA (%) from initial content | 100.4 ± 9.0 |
| NCs Lyo powder | NCs diameter (nm) | 200.2 ± 5.8 |
| following | PDI (nm) | 0.12 ± 0.01 |
| dispersion | CsA content (% w/w) | 5 ± 0.1 |
| Reconstitution | CsA (%) from initial content | 100 ± 2 |
| | Water content (%) | 3.1 ± 0.9 |
| | Free CsA (%) | 7.7 ± 0.9 |
| | Yield (%) | 89.4 ± 0.7 |

It can be noted that the various physicochemical properties were not affected by the lyophilization process and the leakage of CsA from the NCs following lyophilization stress was only 7.7±0.9.

It is important to note that the best batches were yielded by the NCs prepared with the blend of castor oil:labrafil with a moderate advantage to Lactel 100 k E as shown in Table 5.

From the data depicted in Table 5, It can be observed that the total concentration of CsA in the formulation was increased from 5 up to 9%, w/w.

Following lyophilization and reconstitution of the powder, the mean diameter of the NCs increased by 100 nm more or less irrespective of the formulation composition due to the presence of the Kleptose cryoprotectant which surround every NC and protect it from the lyophilization process.

The PDI value is lower than 0.15-0.2 indicative of a good homogeneity of the NC populations especially before lyophilization and after lyophilization and reconstitution of the dispersion, the homogeneity is maintained mainly in the castor oil blend and more particularly with PLGA 100 k.

It is therefore demonstrated that castor oil is able to protect better the NCs from the stress of the lyophilization process than oleic acid and any other oil presented in Table 1 including MCT.

Finally the most promising formulation is the lactel PLGA 100 k castor oil:labrafil at 5% CsA. The 7% formulation can serve as a back-up if needed.

To the best of our understanding, many topical formulations of CsA-loaded nanocarriers have not reached the market because of the limited stability of the nanocarriers in the formulation, and subsequent leakage of the active cargo from the nanocarriers towards the external phase of the topical formulation, resulting in significant damage to the transport efficiency of the active through the skin. Furthermore, NPs of PLGA are water sensitive and may degrade slowly in aqueous formulations. Therefore, they need to be freeze-dried and incorporated within a water-free topical formulation.

The oleic:labrafil-CsA-loaded NCs formulation was chosen in view of the satisfactory results achieved following the lyophilization process (Table1). The NCs were efficiently dispersed in the silicone blend as confirmed by freeze-fracture cryo-SEM depictions [FIG. 1D-D(i)].

This study, thus presented an original design of CsA NCs dispersed in a topical anhydrous formulation ensuring short term stability of CsA in the NCs and probably the same marked at least leakage towards the silicone-based formulation as noted with the lyophilized NC powder.

The topical delivery of CsA using PLGA NCs enhanced its penetration into the viable skin layers and 20% of the initial dose was recovered in the SC layers (FIG. 2). Although the percentage reaching the viable epidermis and dermis was much lower, it was still, to our understanding, at potentially therapeutic tissue levels (FIG. 2). Moreover, other authors also reported that high levels of CsA reached deep layers of the porcine skin using either monoolein as a penetration enhancer, micellar nanocarrier or hydroethanolic solution of skin penetrating peptide. However, to the best of our knowledge, none of these delivery systems have been evaluated in any efficacy study as yet. In this study, at 6- and 24-hour post topical application of the NCs formulation, the concentrations of CsA in the viable epidermis and dermis, were 215 and 260; 11 and 21 ng/mg, respectively. Furlanut et al. reported that in human patients with psoriasis, a CsA concentration higher than 100 ng/ml, at a 12-hour trough is associated with good clinical response (Furlanut et al., 1996). Apparently, the threshold effect is a plausible explanation for the lack of correlation. Indeed, CsA appeared to be concentrated in the skin at levels estimated to be near the peak values in blood (Fisher et al., 1988) and about 10-fold higher than the levels in trough blood samples of patients suffering from plaque-type psoriasis who responded to the treatment (Ellis et al., 1991). We may assume reasonably that skin levels of 1000 ng/g equivalent to 1 ng/mg reported to be active for psoriasis are sufficient to inhibit the activation of inflammatory cells allocated in the skin and involved in AD pathology. The actual levels of CsA in the epidermis and dermis can therefore be considered efficient as previously mentioned. The actual levels of CsA in the epidermis and dermis can be considered efficient. Furthermore, no detectable radioactivity permeation in the receptor fluids through the porcine ear skin could be measured over time, suggesting that very low, if any, radioactivity could traverse the whole skin barrier. Thus, it may be anticipated that possible marked systemic exposure of CsA following topical application is not likely to occur. However, this assumption needs to be confirmed in animal experimentation and more likely in a clinical pharmacokinetic study. Efficacy animal studies were already reported with oleic acid as part of the NC oil core and were submitted previously. However, we were not aware of the marked leakage of CsA following lyophilization. It was therefore important to repeat part of the work with castor oil and compare with oleic to ensure the same efficacy as noted with oleic NCs.

It can be observed from the data presented in FIG. 3 that there is no difference in the permeation profile of CsA in the various layers of the skin between oleic acid or castor oil-based NCs whereas the respective oil solutions did not enhance the skin layers penetration (FIG. 3). It can be assumed that no difference should occur in the efficacy of CsA NCs based on either oleic acid or castor oil core but even an improvement should be expected since significantly less CsA is leaking from the NCs and should even increase the CsA amount penetrating the skin layers and elicit an improved pharmacological activity much needed.

For the purpose of confirming these ex-vivo experimental results, it was decided to carry out also a comparative animal study to validate the conclusions drawn from this ex-vivo experimentation.

TABLE 5
Increasing CsA initial encapsulation content using Oleic acid:Labrafil vs. Castor:Labrafil and different
molecular weight of PLGA. Each batch was triplicated except 8% which was carried as a single batch

| | | | Before lyophilization (aqueous dispersion) | | | | After lyophilization and |
|---|---|---|---|---|---|---|---|
| | | | Mean | | CsA | CsA observed | dispersion reconstitution |
| Oil | | CsA | diameter | | content | (%) from initial | Yield |
| composition | PLGA | (%) | (nm) | PDI value | (% w/w) | concentration | (%) |
| Oleic:labrafil | Purac | 5 | 179.9 ± 23.7 | 0.11 ± 0.01 | 14.1 ± 1.3 | 98.8 ± 8.9 | 92.6 ± 2.9 |
| | 17k E | 7 | 180.6 ± 19.3 | 0.11 ± 0.01 | 16.9 ± 0.7 | 89.6 ± 3.3 | 88.8 ± 1.9 |
| | | 8 | 191.1 | 0.103 | 19.1 | 90.6 | 93.4 |
| | | 9 | 207.2 ± 27.2 | 0.10 ± 0.01 | 19.7 ± 3.4 | 85.3 ± 15.0 | 86.4 ± 5.9 |
| | Lactel | 5 | 165.7 ± 6.6 | 0.11 ± 0.01 | 13.7 ± 0.4 | 96.10 ± 2.9 | 91.2 ± 1.1 |
| | 100k E | 7 | 169.8 ± 7.1 | 0.10 ± 0.01 | 18.6 ± 3.7 | 98.3 ± 19.2 | 90.4 ± 1.1 |
| | | 8 | 162 | 0.121 | 19.8 | 94.2 | 90.3 |
| | | 9 | 172 ± 2.0 | 0.1 ± 0.02 | 22.1 ± 0.7 | 95.4 ± 2.9 | 91.0 ± 0.8 |
| Castor:labrafil | Purac | 5 | 154.5 ± 9.1 | 0.12 ± 0.02 | 13.7 ± 0.4 | 95.7 ± 3.1 | 88.1 ± 20 |
| | 17k E | 7 | 155.7 ± 5.3 | 0.12 ± 0 | 17.3 ± 5.2 | 91.6 ± 2.8 | 89.8 ± 1.4 |
| | | 8 | 153.8 | 0.134 | 18.8 | 89 | 89.3 |
| | | 9 | 160.4 ± 0.7 | 0.12 ± 0.02 | 21.7 ± 0.2 | 93.7 ± 0.6 | 88.5 ± 1.2 |
| | Lactel | 5 | 117.4 ± 12.9 | 0.09 ± 0.01 | 14.3 ± 1.3 | 100.4 ± 9.0 | 89.4 ± 0.7 |
| | 100k E | 7 | 120.9 ± 16.3 | 0.1 ± 0.01 | 18.2 ± 1.3 | 96.2 ± 7.0 | 86.8 ± 3.2 |
| | | 8 | 114.5 | 0.125 | 19.8 | 94.45 | 87.8 |
| | | 9 | 118.9 ± 5.8 | 0.09 ± 0 | 20.1 ± 1.4 | 90.3 ± 5.8 | 89 ± 1.1 |

| | | | After lyophilization and dispersion reconstitution | | | | |
|---|---|---|---|---|---|---|---|
| | | | Mean | | | CsA observed | |
| Oil | | CsA | diameter | | CsA content | (%) from initial | Free CSA |
| composition | PLGA | (%) | (nm) | PDI | (% w/w) | concentration | (% w/w) |
| Oleic:labrafil | Purac | 5 | 286.3 ± 23.1 | 0.19 ± 0.07 | 5.5 ± 0.5 | 110 ± 10 | 16.8 ± 8.6 |
| | 17k E | 7 | 296.5 ± 29.3 | 0.29 ± 0.11 | 6.2 ± 0.8 | 88.6 ± 11.4 | 20.1 ± 11.1 |
| | | 8 | 301 | 0.444 | 7.7 | 96.3 | 21 |
| | | 9 | 294.1 ± 18.0 | 0.29 ± 0.06 | 8.23 ± 1.7 | 91.4 ± 18.9 | 14.9 ± 0.3 |
| | Lactel | 5 | 268.0 ± 16.8 | 0.16 ± 0.05 | 4.8 ± 0.4 | 96 ± 8 | 15.4 ± 3.9 |
| | 100k E | 7 | 265.3 ± 18.1 | 0.20 ± 0.02 | 6.8 ± 0.3 | 97.1 ± 4.3 | 16.4 ± 2.3 |
| | | 8 | 240.3 | 0.205 | 7.77 | 97.1 | 12.44 |
| | | 9 | 272.5 ± 21.5 | 0.18 ± 0.01 | 8.5 ± 1.2 | 94.4 ± 13.3 | 18.4 ± 6.0 |
| Castor:labrafil | Purac | 5 | 221.5 ± 40.4 | 0.18 ± 0.01 | 4.9 ± 0.13 | 98 ± 2.6 | 9.2 ± 5.3 |
| | 17k E | 7 | 236.5 ± 27.3 | 0.18 ± 0.06 | 6.6 ± 0.6 | 94.3 ± 8.6 | 12 ± 6.1 |
| | | 8 | 244.6 | 0.152 | 7.9 | 98.8 | 16.6 |
| | | 9 | 235.7 ± 20.5 | 0.14 ± 0.04 | 7.6 ± 1.5 | 84.4 ± 16.7 | 11.3 ± 3.4 |
| | Lactel | 5 | 200.2 ± 5.8 | 0.12 ± 0.01 | 5 ± 0.1 | 100 ± 2 | 7.7 ± 0.9 |
| | 100k E | 7 | 209.2 ± 17.9 | 0.13 ± 0.02 | 6.9 ± 0.3 | 98.6 ± 4.3 | 7.96 ± 0.4 |
| | | 8 | 201.9 | 0.159 | 7.8 | 97.6 | 6.33 |
| | | 9 | 199.9 ± 7.8 | 0.12 ± 0 | 7.8 ± 0.4 | 86.7 ± 4.4 | 9.2 ± 0.4 |

TABLE 6
Physicochemical data of long-term storage stability at 5 ± 3° C., of lyophilized
NCs prepared under similar conditions as a function of castor oil or oleic acid core.

| | | 0 m | 1 m | 3 m | 6 m | 9 m |
|---|---|---|---|---|---|---|
| | Formulation | 16.6.16 | 16.7.16 | 16.9.16 | 16.12.16 | 16.3.17 |
| Appearance | 1 O:L 5% 100K | White powder | White powder | White powder | White powder | |
| | 2 O:L 5% 17K | White powder | White powder | White powder | White powder | |
| | 3 L:C 5% 100K | White powder | White powder | White powder | White powder | |
| | 4 L:C 5% 17K | White powder | White powder | White powder | White powder | |
| | 5 O:L 7% 17K | White powder | White powder | White powder | White powder | |
| | 6 O:L 7% 100K | White powder | White powder | White powder | White powder | |
| | 7 L:C 7% 17K | White powder | White powder | White powder | White powder | |
| | 8 L:C 7% 100K | White powder | White powder | White powder | White powder | |
| Size/PDI | 1 O:L 5% 100K | 278.4 ± 1.015 | 279.9 ± 9.722 | 265.6 ± 1.422 | 261.3 ± 4.751 | |
| (nm) | | 0.166 ± 0.024 | 0.210 ± 0.026 | 0.111 ± 0.018 | 0.215 ± 0.040 | |
| | 2 O:L 5% 17K | 283.3 ± 2.946 | 295.8 ± 5.103 | 273.1 ± 6.689 | 283.2 ± 1.553 | |
| | | 0.160 ± 0.017 | 0.197 ± 0.028 | 0.189 ± 0.008 | 0.191 ± 0.015 | |
| | 3 L:C 5% 100K | 198.5 ± 0.923 | 201.4 ± 3.559 | 203.1 ± 0.757 | 199.9 ± 2.203 | |
| | | 0.122 ± 0.048 | 0.155 ± 0.033 | 0.080 ± 0.033 | 0.120 ± 0.028 | |
| | 4 L:C 5% 17K | 208.1 ± 1.480 | 210.7 ± 8.240 | 210 ± 3.029 | 204.4 ± 0.929 | |
| | | 0.122 ± 0.029 | 0.172 ± 0.038 | 0.137 ± 0.019 | 0.142 ± 0.045 | |
| | 5 O:L 7% 17K | 263.7 ± 1.480 | 265.6 ± 4.329 | 250.7 ± 1.550 | 321.2 ± 10.49 | |
| | | 0.159 ± 0.022 | 0.200 ± 0.025 | 0.166 ± 0.007 | 0.256 ± 0.065 | |
| | 6 O:L 7% 100K | 269.1 ± 2.108 | 256.3 ± 6.834 | 260.9 ± 4.678 | 270.8 ± 2.829 | |
| | | 0.174 ± 0.046 | 0.209 ± 0.047 | 0.196 ± 0.049 | 0.161 ± 0.025 | |
| | 7 L:C 7% 17K | 225.4 ± 0.776 | 226.1 ± 2.810 | 222 ± 3.509 | 219.8 ± 3.691 | |
| | | 0.139 ± 0.029 | 0.155 ± 0.031 | 0.151 ± 0.013 | 0.161 ± 0.013 | |
| | 8 L:C 7% 100K | 212.1 ± 3.201 | 215.6 ± 0.586 | 210.2 ± 2.454 | 211.5 ± 2.303 | |
| | | 0.093 ± 0.023 | 0.144 ± 0.030 | 0.137 ± 0.031 | 0.101 ± 0.026 | |
| % Water | 1 O:L 5% 100K | 3.7 | 4.0 | 3.0 | 1.9 | |
| content | 2 O:L 5% 17K | 3.6 | 4.2 | 3.2 | 1.2 | |
| | 3 L:C 5% 100K | 2.8 | 4.1 | 3.0 | 0.1 | |
| | 4 L:C 5% 17K | 3.0 | 3.9 | 3.2 | 1.9 | |
| | 5 O:L 7% 17K | 2.9 | 4.2 | 3.4 | 3.8 | |
| | 6 O:L 7% 100K | 2.5 | 3.5 | 3.0 | 2.0 | |
| | 7 L:C 7% 17K | 3.0 | 4.2 | 3.5 | 0.5 | |
| | 8 L:C 7% 100K | 2.7 | 3.5 | 3.0 | 2.3 | |
| % Free | 1 O:L 5% 100K | 11.7 | 11.2 | 12.7 | 12.9 | |
| CsA | 2 O:L 5% 17K | 15.6 | 16.0 | 16.2 | 17.7 | |
| | 3 L:C 5% 100K | 4.5 | 5.5 | 5.4 | 5.8 | |
| | 4 L:C 5% 17K | 6.2 | 6.9 | 7.0 | 7.8 | |
| | 5 O:L 7% 17K | 12.4 | 12.8 | 13.3 | 12.5 | |
| | 6 O:L 7% 100K | 14.0 | 13.5 | 13.7 | 20.6 | |
| | 7 L:C 7% 17K | 7.8 | 8.0 | 9.4 | 1.8 | |
| | 8 L:C 7% 100K | 6.6 | 7.6 | 6.5 | 16.2 | |
| CsA | 1 O:L 5% 100K | 4.5 | 4.7 | 5.3 | 4.8 | |
| content | 2 O:L 5% 17K | 4.7 | 4.9 | 5.4 | 5.0 | |
| (%, W/W) | 3 L:C 5% 100K | 4.5 | 4.8 | 5.0 | 4.7 | |
| | 4 L:C 5% 17K | 4.8 | 4.4 | 5.0 | 4.6 | |
| | 5 O:L 7% 17K | 5.8 | 6.3 | 6.7 | 6.2 | |
| | 6 O:L 7% 100K | 5.1 | 6.4 | 7.0 | 6.5 | |
| | 7 L:C 7% 17K | 6.3 | 7.6 | 7.0 | 6.5 | |
| | 8 L:C 7% 100K | 6.3 | 6.2 | 7.1 | 6.5 | |

TABLE 7
Physicochemical data of long-term storage stability at 25 ± 3° C., of lyophilized
NCs prepared under similar conditions as a function of castor oil or oleic acid core.

| | | 0 m | 1 m | 3 m | 6 m | 18 m |
|---|---|---|---|---|---|---|
| | Formulation | 19.5.16 | 16.6.16 | 19.8.16 | 19.11.16 | 19.11.17 |
| Appearance | 1 O:L 5% 100K | White powder | White powder | White powder | White powder | |
| | 2 O:L 5% 17K | White powder | White powder | White powder | White powder | |
| | 3 L:C 5% 100K | White powder | White powder | White powder | White powder | |
| | 4 L:C 5% 17K | White powder | White powder | White powder | White powder | |
| | 5 O:L 7% 17K | White powder | White powder | White powder | White powder | |
| | 6 O:L 7% 100K | White powder | White powder | White powder | White powder | |
| | 7 L:C 7% 17K | White powder | White powder | White powder | White powder | |
| | 8 L:C 7% 100K | White powder | White powder | White powder | White powder | |
| Size/PDI | 1 O:L 5% 100K | 254.1 ± 5.880 | 278.4 ± 1.015 | 274.7 ± 2.250 | 287.7 ± 6.854 | |
| (nm) | | 0.113 ± 0.023 | 0.166 ± 0.024 | 0.150 ± 0.055 | 0.152 ± 0.032 | |
| | 2 O:L 5% 17K | 269.9 ± 2.858 | 283.3 ± 2.946 | 277.7 ± 3.729 | 294.3 ± 4.359 | |
| | | 0.144 ± 0.015 | 0.160 ± 0.017 | 0.171 ± 0.035 | 0.178 ± 0.005 | |
| | 3 L:C 5% 100K | 178.0 ± 0.8963 | 198.5 ± 0.923 | 272.3 ± 3.083 | 213.5 ± 1.305 | |
| | | 0.178 ± 0.036 | 0.122 ± 0.048 | 0.130 ± 0.075 | 0.107 ± 0.031 | |
| | 4 L:C 5% 17K | 178.3 ± 0.5508 | 208.1 ± 1.480 | 211.1 ± 1.069 | 213.9 ± 2.352 | |
| | | 0.173 ± 0.006 | 0.122 ± 0.029 | 0.152 ± 0.049 | 0.103 ± 0.024 | |
| | 5 O:L 7% 17K | 262.9 ± 6.465 | 263.7 ± 1.480 | 273.3 ± 7.778 | 280.1 ± 5.424 | |
| | | 0.216 ± 0.010 | 0.159 ± 0.022 | 0.194 ± 0.021 | 0.181 ± 0.031 | |
| | 6 O:L 7% 100K | 253.6 ± 9.260 | 269.1 ± 2.108 | 266.5 ± 1.300 | 269.3 ± 1.637 | |
| | | 0.224 ± 0.010 | 0.174 ± 0.046 | 0.222 ± 0.069 | 0.093 ± 0.027 | |
| | 7 L:C 7% 17K | 216.9 ± 2.325 | 225.4 ± 0.776 | 273.2 ± 7.580 | 229.3 ± 2.203 | |
| | | 0.291 ± 0.023 | 0.139 ± 0.029 | 0.185 ± 0.008 | 0.136 ± 0.001 | |
| | 8 L:C 7% 100K | 196.1 ± 2.838 | 212.1 ± 3.201 | 213.3 ± 5.320 | 212.9 ± 2.150 | |
| | | 0.115 ± 0.022 | 0.093 ± 0.023 | 0.116 ± 0.019 | 0.106 ± 0.023 | |
| % Water | 1 O:L 5% 100K | 5.1 | 3.7 | 4.2 | 4.2 | |
| content | 2 O:L 5% 17K | 4.7 | 3.6 | 4.2 | 5.4 | |
| | 3 L:C 5% 100K | 4.2 | 2.8 | 4.4 | 5.1 | |
| | 4 L:C 5% 17K | 5.2 | 3.0 | 4.1 | 5.4 | |
| | 5 O:L 7% 17K | 4.8 | 2.9 | 4.1 | 5.4 | |
| | 6 O:L 7% 100K | 2.6 | 2.5 | 3.5 | 5.0 | |
| | 7 L:C 7% 17K | 5.0 | 3.0 | 3.7 | 5.3 | |
| | 8 L:C 7% 100K | 2.3 | 2.7 | 3.8 | 5.6 | |
| % Free | 1 O:L 5% 100K | 11.5 | 11.7 | 12.9 | 11.6 | |
| CsA | 2 O:L 5% 17K | 10.7 | 15.6 | 17.8 | 13.7 | |
| | 3 L:C 5% 100K | 5.4 | 4.5 | 7.08 | 5.3 | |
| | 4 L:C 5% 17K | 5.4 | 6.2 | 5.45 | 6.9 | |
| | 5 O:L 7% 17K | 12.2 | 12.4 | 10.7 | 18.7 | |
| | 6 O:L 7% 100K | 14.9 | 14.0 | 10.2 | 18.5 | |
| | 7 L:C 7% 17K | 7.7 | 7.8 | 10.7 | 10.7 | |
| | 8 L:C 7% 100K | 8.2 | 6.6 | 9.2 | 8.9 | |
| CsA | 1 O:L 5% 100K | 4.9 | 4.5 | 4.9 | 5.3 | |
| content | 2 O:L 5% 17K | 5.3 | 4.7 | 4.9 | 5.1 | |
| (%, W/W) | 3 L:C 5% 100K | 5.0 | 4.5 | 4.6 | 5.0 | |
| | 4 L:C 5% 17K | 5.0 | 4.8 | 4.6 | 4.7 | |
| | 5 O:L 7% 17K | 6.8 | 5.8 | 6.1 | 6.3 | |
| | 6 O:L 7% 100K | 7.1 | 5.1 | 6.5 | 6.4 | |
| | 7 L:C 7% 17K | 7.0 | 6.3 | 8.3 | 6.7 | |
| | 8 L:C 7% 100K | 7.2 | 6.3 | 6.3 | 7.2 | |

TABLE 8
Physicochemical data of long-term storage stability at 37° C., of lyophilized
NCs prepared under similar conditions as a function of castor oil or oleic acid core.

| | | 0 m | 2 weeks | 1 m | 3 m | 6 m |
|---|---|---|---|---|---|---|
| | Formulation | 16.6.16 | 30.6.16 | 16.7.16 | 16.9.16 | 16.12.16 |
| Appearance | 1 O:L 5% 100K | White powder | White powder | White powder | White powder | White powder |
| | 2 O:L 5% 17K | White powder | White powder | White powder | White powder | White powder |
| | 3 L:C 5% 100K | White powder | White powder | White powder | White powder | White powder |
| | 4 L:C 5% 17K | White powder | White powder | White powder | White powder | White powder |
| | 5 O:L 7% 17K | White powder | White powder | White powder | White powder | White powder |
| | 6 O:L 7% 100K | White powder | White powder | White powder | White powder | White powder |
| | 7 L:C 7% 17K | White powder | White powder | White powder | White powder | White powder |
| | 8 L:C 7% 100K | White powder | White powder | White powder | White powder | White powder |
| Size/PDI | 1 O:L 5% 100K | 278.4 ± 1.015 | 279.1 ± 11.41 | 282.7 ± 3.970 | 265.5 ± 0.6028 | 269.4 ± 5.839 |
| (nm) | | 0.166 ± 0.024 | 0.191 ± 0.014 | 0.207 ± 0.036 | 0.177 ± 0.028 | 0.178 ± 0.016 |
| | 2 O:L 5% 17K | 283.3 ± 2.946 | 225.3 ± 46.83 | 295.6 ± 5.217 | 283.8 ± 0.300 | 256.9 ± 7.425 |
| | | 0.160 ± 0.017 | 0.117 ± 0.067 | 0.214 ± 0.004 | 0.163 ± 0.018 | 0.144 ± 0.074 |
| | 3 L:C 5% 100K | 198.5 ± 0.923 | 202.2 ± 6.374 | 203.5 ± 2.194 | 198 ± 1.150 | 141.2 ± 2.318 |
| | | 0.122 ± 0.048 | 0.130 ± 0.05 | 0.131 ± 0.022 | 0.096 ± 0.030 | 0.122 ± 0.016 |
| | 4 L:C 5% 17K | 208.1 ± 1.480 | 247.6 ± 32.6 | 217.0 ± 3.899 | 209.7 ± 0.6807 | 213.8 ± 0.8386 |
| | | 0.122 ± 0.029 | 0.168 ± 0.048 | 0.148 ± 0.030 | 0.120 ± 0.021 | 0.113 ± 0.023 |
| | 5 O:L 7% 17K | 263.7 ± 1.480 | 263.6 ± 4.180 | 268.4 ± 5.510 | 255.4 ± 2.914 | 286.0 ± 5.752 |
| | | 0.159 ± 0.022 | 0.190 ± 0.037 | 0.221 ± 0.027 | 0.156 ± 0.010 | 0.203 ± 0.021 |
| | 6 O:L 7% 100K | 269.1 ± 2.108 | 239.5 ± 25.44 | 269.7 ± 4.917 | 273.9 ± 3.625 | 277.8 ± 2.721 |
| | | 0.174 ± 0.046 | 0.122 ± 0.031 | 0.245 ± 0.014 | 0.158 ± 0.043 | 0.219 ± 0.006 |
| | 7 L:C 7% 17K | 225.4 ± 0.776 | 215.2 ± 5.160 | 231.7 ± 5.859 | 225.2 ± 4.165 | 231.7 ± 1.212 |
| | | 0.139 ± 0.029 | 0.077 ± 0.059 | 0.167 ± 0.031 | 0.113 ± 0.013 | 0.141 ± 0.026 |
| | 8 L:C 7% 100K | 212.1 ± 3.201 | 211.6 ± 2.778 | 215.2 ± 6.214 | 208.3 ± 2.879 | 212.7 ± 1.682 |
| | | 0.093 ± 0.023 | 0.067 ± 0.050 | 0.157 ± 0.027 | 0.100 ± 0.025 | 0.101 ± 0.018 |
| % Water | 1 O:L 5% 100K | 3.75 | 3.0 | 3.6 | 1.5 | 2.1 |
| content | 2 O:L 5% 17K | 3.6 | 2.9 | 3.9 | 3.3 | 0.7 |
| | 3 L:C 5% 100K | 2.8 | 2.8 | 3.6 | 2.9 | 1.6 |
| | 4 L:C 5% 17K | 3.0 | 2.15 | 3.0 | 2.7 | 2.2 |
| | 5 O:L 7% 17K | 2.9 | 2.0 | 3.0 | 3.3 | ND |
| | 6 O:L 7% 100K | 2.5 | 2.1 | 2.8 | 2.4 | ND |
| | 7 L:C 7% 17K | 3.0 | 2.3 | 3.0 | 2.5 | ND |
| | 8 L:C 7% 100K | 2.7 | 2.0 | 3.0 | 2.3 | ND |
| % Free | 1 O:L 5% 100K | 11.7 | 12.6 | 11.6 | 12.6 | 11.4* |
| CsA | 2 O:L 5% 17K | 15.6 | 15.5 | 19.6 | 15.7 | 9.2* |
| | 3 L:C 5% 100K | 4.5 | 4.0 | 4.8 | 5.7 | 5.3 |
| | 4 L:C 5% 17K | 6.2 | 6.0 | 6.8 | 7.1 | 6.4 |
| | 5 O:L 7% 17K | 12.4 | 12.8 | 17.6 | 13.4 | 17.6* |
| | 6 O:L 7% 100K | 14.0 | 13.0 | 18.2 | 15.0 | 18.7* |
| | 7 L:C 7% 17K | 7.8 | 7.0 | 10.8 | 9.7 | 12.5 |
| | 8 L:C 7% 100K | 6.6 | 5.9 | 9.2 | 6.9 | 8.1 |
| CsA | 1 O:L 5% 100K | 4.5 | 5.2 | 5.2 | 5.1 | 4.4* |
| content | 2 O:L 5% 17K | 4.7 | 4.7 | 4.9 | 4.7 | 3.0* |
| (%, W/W) | 3 L:C 5% 100K | 4.5 | 4.8 | 4.5 | 5.1 | 4.5 |
| | 4 L:C 5% 17K | 4.8 | 4.3 | 5.1 | 5.1 | 4.6 |
| | 5 O:L 7% 17K | 5.8 | 6.5 | 6.3 | 6.9 | 5.8* |
| | 6 O:L 7% 100K | 5.1 | 6.5 | 6.2 | 7.1 | 6.4* |
| | 7 L:C 7% 17K | 6.3 | 7.15 | 6.8 | 7.1 | 6.5 |
| | 8 L:C 7% 100K | 6.3 | 6.8 | 6.3 | 6.9 | 6.3 |

Contact Hypersensitivity (CHS) Mice Model

Induction of CHS was performed as described below. Four days before CHS sensitization the 6-7 week-old BALB/c mice abdomens were carefully shaved and allowed to rest for recovery. On the day of sensitization, various topical CsA formulations and Protopic® were applied to the shaved skin (20 mg of either Ca:La or Ol:La CsA NCs and empty NCs semisolid anhydrous preparation, all equivalent to 20 μg/cm² CsA). Four hours after topical treatments, to elicit CHS, mice were sensitized with 50 μl 1% oxazolone in acetone/olive oil (AOO) 4:1 on the shaved abdomen. They were challenged five days later with 25 μl 0.5% oxazolone in AOO on the back of the right ear only. The left ear was untreated and swelling responses were measured by micrometer (Mytutoyo, USA), recording the difference between left and right ears at 24, 48, 72, 96 and 168 hours after challenge. The average swelling of 150 μm was considered an allergic reaction.

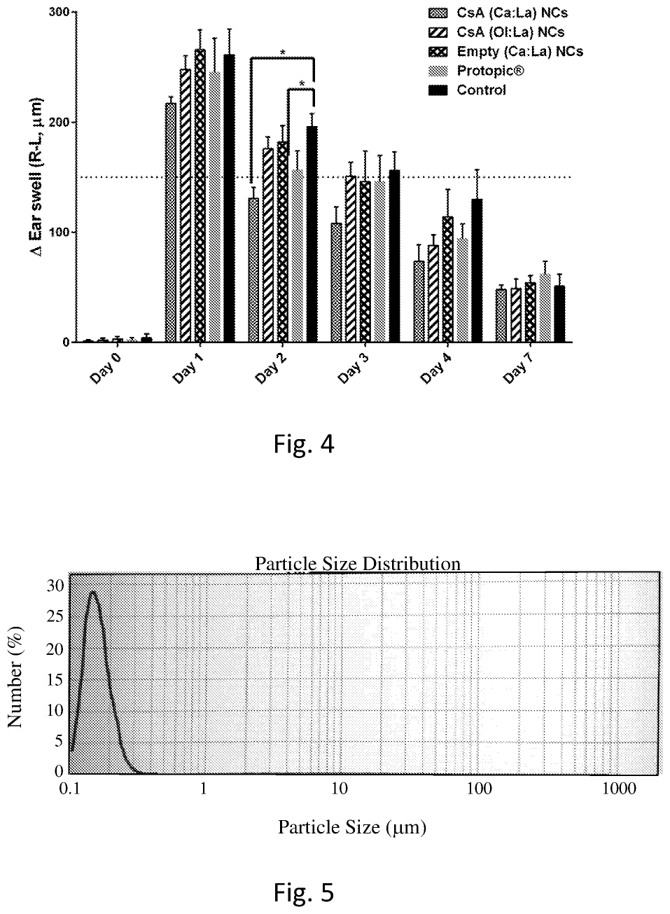

It can be noted that castor oil based CsA NCs are as effective as the oleic acid based NCs. It can further be observed that at day 2 (FIG. 4), Castor oil based NCs elicited a significant improved effect than oleic acid based CsA NCs confirming the previous deductions.

More importantly, it was also observed that the long-term stability of CsA NCs was much more in favor of the castor oil than the oleic acid as shown in the results presented in Tables 6-8.

Only with the castor oil core the various parameters were stable especially over 6 months at 37° C.

These results clearly indicate that only with castor oil, it will be possible to design a product for the market since, a stability of 6 months at 37° C. is equivalent to a shelf life of the commercial product of 3 years whereas such a stable product cannot be achieved with oleic acid as shown in Tables 6-8.

Ocular Delivery

Background

The human eye is a complex organ that consists of many different cell types. Topical administration of drugs remains the preferred route for the treatment of ocular diseases primarily because of the ease of application and patient compliance. However, the absorption of topically applied drugs to the eyes is very poor because of the inherent anatomical and physiological barriers leading to the requirement for repeated high-dose administrations. Firstly, drug molecules are diluted on the precorneal tear film, with an approximate total thickness of 10 μm. The rapid renewal rate of the outer layers of this lachrymal fluid (1-3 μl/min) together with the blinking reflex, severely limits the residence time of drugs in the precorneal space (<1 min) and, thus, the ocular bioavailability of the instilled drugs (<5%). Depending on the target sites of the different ocular pathologies, drugs either need to be retained at the cornea and/or conjunctiva or cross these barriers and reach the internal structures of the eye. The entry of drugs through the conjunctiva is normally associated with systemic drug absorption and it is highly impeded by the sclera. As a consequence, the cornea represents the main route of access for drugs whose target is in the inner eye. Unfortunately, crossing the corneal barrier represents a key challenge for many drugs. Indeed, the multilayer lipophilic corneal epithelium is highly organized with the presence of abundant tight junctions and desmosomes that effectively exclude foreign molecules and particles. Moreover, the hydrophilic stroma makes the transport of drugs very difficult. Only drugs with a low molecular weight and a moderate lipophilic character can deal with these barriers and only in a modest manner.

Vernal keratoconjunctivitis (VKC) is a bilateral, chronic sight-threatening and severe inflammatory ocular disease mainly occurring in children. The common age of onset is before 10 years (4-7 years of age). A male preponderance has been observed, especially in patients under 20 years of age, among whom the male:female ratio is 4:1-3:1. Although vernal (spring) implies a seasonal predilection of the disease, its course commonly occurs mostly year round, particularly in the tropics. VKC can be found throughout the world and has been reported from almost all continents. Atopic sensitization has been found in around 50% of patients. Patients with VKC usually present primarily with eye symptoms, the more predominant being itching, discharge, tearing, eye irritation, redness of the eyes, and to variable extent, photophobia.

VKC has been included in the newest classification of ocular surface hypersensitivity disorders as both an IgE- and non-IgE-mediated ocular allergic disease. Nonetheless, it is also well known that not all VKC patients have positive allergy skin tests. The increased numbers of Th2 lymphocytes in the conjunctiva and the increased expression of co-stimulatory molecules and cytokines suggest that T cells play a crucial role in the development of VKC3. In addition, to typical Th2-derived cytokines, Th1-type cytokines, pro-inflammatory cytokines, a variety of chemokines, growth factors, and enzymes are overly expressed in VKC patients.

### 1. VKC Treatment

Common therapies include topical antihistamines and mast cell stabilizers. These therapies are infrequently sufficient and topical corticosteroids are often required for the treatment of exacerbations and more severe cases of the disease. Corticosteroids remain the mainstay therapy of the ocular inflammation acting as both anti-inflammatory and immunosuppressive drugs. The goal of therapy is to prevent ocular damage, scarring and ultimately vision loss. While these agents are very effective, they are not without associated risks. The ocular side effects of long term steroid use for all types and means of administration include cataract formation, increased intraocular pressure and higher susceptibility to infections. In order to overcome the potentially blinding complications of topical steroids, immunomodulatory drugs such as Cyclosporine A and Tacrolimus are being used more frequently.

Tacrolimus was efficient as a steroid sparing agent even in severe cases of VKC which were refractory to Cyclosporine.

### 2. Tacrolimus Efficacy and Limitations

Tacrolimus, also known as FK506, is a macrolide produced from the fermentation broth of Japanese soil sample that contained the bacteria Streptomyces tsukubaensis. This drug binds to FK506-binding proteins within T lymphocytes and inhibits calcineurin activity. Calcineurin inhibition suppresses dephosphorylation of the nuclear factor of activated T cells and its transfer into the nucleus, which results in the suppressed formation of cytokines by T lymphocytes. Inhibition of T lymphocytes may therefore lead to the inhibition of release of inflammatory cytokines and decreased stimulation of other inflammatory cells. The immunosuppressive effects of Tacrolimus are not limited to T lymphocytes, but it may also act on B cells, neutrophils and mast cells leading to improvement of symptoms and signs of VKC.

Different forms and concentrations of tacrolimus have been assessed in the treatment of anterior segment inflammatory disorders. The main concentration of topical tacrolimus formulations that was investigated in the majority of the clinical trials was 0.1%. Some other studies evaluated lower concentrations of tacrolimus including 0.005, 0.01, 0.02 and 0.03% and showed that topical eye drop was a safe and effective treatment modality for patients with VKC refractory to conventional medications including topical steroids. However, Tacrolimus has difficulty penetrating the corneal epithelium and accumulates in the corneal stroma due to its poor water solubility and relatively high molecular weight. Moreover, there is no worldwide ophthalmic marketed formulation of this drug, obliging patients with VKC to use a dermatologic Tacrolimus ointment meant to treat atopic dermatitis.

### 3. Nanocarriers for the Treatment of Ocular Diseases

Development of an efficient topical dosage form that is capable of delivering the drug at the correct dose without the need for frequent instillation represents a major challenge for pharmaceutical sciences and technology. In the last decades, it has been shown that specific nanocarriers with size <1000 nm can overcome the eye-associated barriers. Indeed, they have shown the capacity to associate a wide variety of drugs, including highly lipophilic drugs, reduce the degradation of labile drugs, increase the residence time of the associated drugs onto the ocular surface and improve their interaction with the corneal and conjunctival epithelia and consequently their bioavailability. Nanocolloidal systems include liposomes, nanoparticles and nanoemulsions.

### 3.1. Polymeric Nanoparticles

Polymeric nanoparticles (PNs) are colloidal carriers with diameters ranging from 10 to 1000 nm and comprise various biodegradable and non-biodegradable polymers. PNs can be classified as nanospheres (NSs) or nanocapsules (NCs); NSs are matrix systems that adsorb or entrap a drug whereas NCs are reservoir-type systems with a surrounding polymeric wall containing an oil core where the drug is dispersed.

These systems have been studied as topical ocular delivery systems and showed enhanced adherence to the ocular surface and their controlled release of drugs. Because these PNs can mask the physico-chemical properties of the entrapped drugs, they can improve drug stability and consequently improve drug bioavailability. In addition, these colloidal carriers can be administered in liquid form, facilitating administration and patient compliance.

Nanoemulsions (NEs) are heterogeneous dispersions of two immiscible liquids (oil-in-water or water-in-oil) stabilized by the use of surfactants. These homogeneous systems are all fluids of low viscosity, thus applicable for topical administration to the eyes. Moreover, presence of surfactants increases membrane permeability, thereby increasing drug uptake. In addition to this, NEs provide sustained release of drugs and have the capacity to accommodate both hydrophilic and lipophilic drugs. In light of the numerous advantages of nanocarriers in topical eye delivery and the already proved efficiency of Tacrolimus in Vernal keratoconjunctivitis, our research focused on the development

In this study, it is hypothesized that Tacrolimus encapsulation in colloidal delivery systems (Nanocapsules and/or Nanoemulsions) will improve the corneal drug retention and increase its ocular penetration, resulting in a higher therapeutic effect in VKC.

The overall objective is to develop a stable, colloidal ophthalmic formulation loaded with Tacrolimus to fulfill the need of a worldwide commercially available treatment for refractory VKC patients.

In this study, we focused on the following aims:
a—Design of Tacrolimus nanocarriers (NEs/NCs) and their characterization
b—Formulations' stabilization and adaptation to the physiologic conditions of the eyes
c—Ex-vivo evaluation of the nanocarriers' pig cornea penetration and ex-vivo toxicity assessment of selected nanocarriers on excised pig corneas.

### 4. Materials

Tacrolimus (as monohydrate) was kindly donated by TEVA (Opava, Komárov, Czech Republic); Castor oil was acquired from TAMAR industries (Rishon LeTsiyon, Israel), Polysorbate 80 (Tween® 80), Polyoxyl-35 castor oil (CremophorEL), D (+) Trehalose, D-Mannitol, Sucrose, MTT (3-(4,5-dimethylthiazol-2-yl)-2,5-diphenyltetrazolium bromide) were purchased from Sigma-Aldrich (Rehovot, Israel). Lipoid E80 was acquired from Lipoid GmbH (Ludwigshafen, Germany) and Middle chain triglyceride (MCT) was kindly provided by Societe des Oleagineux (Bougival, France). Glycerin was acquired from Romical (Be'er-Sheva, Israel). [³H]-Tacrolimus, Ultima-Gold® liquid scintillation cocktail and Solvable® were purchased from Perkin-Elmer (Boston, Mass., USA). PVA (Mowiol 4-88) was acquired from Efal Chemical Industries (Netanya, Israel); PLGA 4.5K (MW: 4.5 KDa), PLGA 7.5K (MW: 7.5 KDa) and PLGA 17K (MW: 17 KDa) were acquired from Evonik Industries (Essen, Germany). PLGA 50 K (MW 50 KDa) was purchased from Lakeshore Biomaterials (Birmingham, Ala., USA) and PLGA 100K (MW 100 KDa) from Lactel® ‘ (Durect Corp., AL, USA). Macrogol 15 hydroxystearate (Solutol® ’ HS 15) was kindly donated by BASF (Ludwigshafen, Germany). (2-Hydroxypropyl)β-cyclodextrin (HPBCD) was from Carbosynth (Compton, UK). All organic solvents were HPLC grade and purchased from J. T Baker (Deventer, Holland). All tissue culture products were from Biological Industries Ltd. (Beit Ha Emek, Israel).

### 5. Methods

5.1. Preparation of the Nanocarriers

5.1.1. Preparation of Blank and Drug-Loaded NPs

The various PLGA nanoparticles were prepared according to the well-established solvent displacement method²⁰. Briefly, the polymer poly lactic-co-glycolic acid (PLGA) at (50:50 blend of lactic acid:glycolic acid), was dissolved in acetone at a concentration of 0.6% w/v. For NCs preparation, MCT/castor oil and Tween 80/Cremophor EL/Lipoid E80, were introduced to the organic phase in diverse concentrations and combinations, with the aim of formulations scanning. For NSs preparation, no oil was mixed to the organic phase. Tacrolimus was added to the organic phase at several concentrations, which the optimums were 0.05 and 0.1% w/v. The organic phase was poured into the aqueous phase which contained 0.2-0.5% w/v Solutol® HS 15 or 1.4% w/v PVA. The volume ratio between the organic and aqueous phases was 1:2 v/v. The suspension was stiffed at 900 rpm for 15 min and then all acetone was removed by reduced pressure evaporation. For a concentrated formulation, water was also vaporized until the desired final volume was achieved. Purification of the NPs was performed by centrifugation (4000 rpm; 5 min; 25° C.). In order to achieve optimal formulations for Tacrolimus, many NPs and particularly NCs formulations were prepared, enabling us to determine the effects of PLGA MW, active ingredient concentration, oil types and the presence of different surfactants in aqueous and organic phase on NP's stability and properties.

5.1.2. Preparation of Drug-Loaded NEs

The different nanoemulsions were prepared by the same process described for the NCs without addition of the polymer PLGA. These formulations were further diluted with water to attain the goal of tacrolimus concentration at 0.05% w/v.

When radiolabeled NCs/NEs were prepared, 3 μCi of [³H]-Tacrolimus was mixed with 0.05% w/v of Tacrolimus acetone solution before addition to the aqueous phase.

5.2. Physicochemical Characterization of the Nanocarriers

5.2.1. Particle/Droplet-Size Measurements

5.2.1.1. Zetasizer Nano ZS

Mean diameter of the various NCs and NEs were measured by Malvern's Zetasizer instrument (Nano series, Nanos-ZS) at 25° C. 10 μL of each formulation was diluted in 990 μL water for HPLC.

5.2.1.2. Mastersizer

NEs' droplets sizes were also measured by using a Mastersizer 2000 (Malvern Instruments, UK). Approximately 5 mL of each NE was used per measurement, dispersed in 120 ml of DDW, and measured under constant stirring (˜1,760 rpm).

5.2.2. Morphological Evaluation

5.2.2.1. Transmission Electron Microscopy (TEM) Imaging

Transmission electron microscopy (TEM) observations were evaluated using a JEM-1400plus 120 kV (JEOL Ltd.). Specimens were prepared by mixing the samples with uranyl acetate for negative staining.

5.2.2.2. Cryo-Transmission Electron Microscopy (Cryo-TEM) Imaging

For cryo-transmission electron microscopy (Cryo-TEM) observations, a drop of NEs/NPs suspension was placed on carbon-coated perforated polymer film supported on a 300 mesh Cu grid (Ted Pella Ltd.) and the specimen was automatically vitrified using Vitrobot Mark-IV (FEI), by means of a fast quench in liquid ethane to −170° C. The samples were studied using Tecnai T12 G2 Spirit TEM (FEI), at 120 kV with a Gatan cryo-holder maintained at −180° C.

5.3 Lyophilization of the NPs

Some cryoprotectants were tested in various mass ratios ranging from 1:20 to 1:1 (PLGA:cryoprotectant). One part of the aqueous solution of cryoprotectants was added to one part of the fresh NPs suspension and mixed well. Preparations were then lyophilized for 17 h by Epsilon 2-6D freeze-drier (Christ). When needed, an amount of dried powder, equivalent to calculated weight of 1 mL NPs, was dispersed in 1 mL of water to reconstitute the initial dispersion, and the reconstitution was characterized by particle-size distribution.

5.4. Isotonicity Adjustment and Measurement

To achieve isotonicity, glycerin was added to the different formulations. For NEs and fresh NPs, a concentration of 2.25% w/v glycerin was needed, whereas for lyophilized and reconstituted NPs, 2% w/v were sufficient. Osmolality measurements were performed on 3MO Plus Micro Osmometer (Advanced Instruments Inc., Massachusetts, USA).

5.5. Tacrolimus Quantification

5.5.1. Drug Content in NEs/Fresh NPs

The Tacrolimus content (in weight/volume) in NEs was determined by HPLC. 50 μl of the NEs were added to 950 μl of acetonitrile and were injected into an HPLC system equipped with UV detector (Dionex ultimate 300, Thermo Fisher Scientific). Using a 5 μm Phenomenex C18 column (4.6×150 mm) (Torrance, Calif., USA), a flow rate of 0.5 mL/min at 60° C. and a 95:5 v/v mixture of acetonitrile:water as mobile phase, Tacrolimus was detected at the wavelength of 213 nm, with a retention time of 5.1 min.

5.5.2. Drug Loading in Lyophilized NPs

20 mg of lyophilized NPs were reconstituted in 2.5 mL of water and further sonicated for 10 min. 1 mL of this dispersion was then added to 9 mL of Acetonitrile and vortexed during five minutes. The loading efficiency of Tacrolimus in lyophilized NPs was determined by HPLC. 1 mL of the latter solution was injected into the HPLC system described previously. Tacrolimus loading in the lyophilized powder was determined as described in equation (1).

$$\% Tac ( w \text{/} w ) = \frac{Drug amount}{Lyophilized powder amount} \qquad ( 1 )$$

5.6. Tacrolimus NPs Encapsulation Efficiency Assay

For encapsulation efficiency (EE) determination of fresh NPs, 1 mL formulation was placed in 1.5 mL caped polypropylene tube (Beckman Coulter) and ultra-centrifuged at 45000 rpm for 75 min at 4° C. (Optima MAX-XP ultracentrifuge, TLA-45 Rotor, Beckman Coulter). Supernatant was separated for HPLC analysis. Free Tacrolimus amount was determined by dissolving 100 μL of supernatant in 900 μL acetonitrile. EE was calculated according to equation (2).

$$EE ( \% ) = \frac{Initial amount of drug - Free amount of drug}{Initial amount of drug} * 100 \qquad ( 2 )$$

For Encapsulation efficiency determination of lyophilized NPs, 8 mg of the lyophilized powder were reconstituted in 1 mL of water and ultra-centrifuged at the speed of 40000 rpm for 40 min at 4° C. Encapsulation efficiency was determined as previously described for fresh NPs.

5.7. Tacrolimus Loaded Nanocarriers Stability Assay

5.7.1. Stability Evaluation of NEs

Fresh Tacrolimus NEs were divided in samples of 1 mL which were kept sealed at 4° C., Room Temperature and 37° C. and protected from light. NEs stability was evaluated at 1, 2, 4, and 8 weeks by taking a sample for droplet size distribution and drug content using the same protocol previously described.

5.7.2. Stability Evaluation of NPs

Tacrolimus NPs dried-powder was divided into samples of 150 mg which were kept sealed at 4° C., Room Temperature and 37° C. and protected from light. The powder was analyzed at 1, 2, 4, 8, 12 and 17 weeks. At the end of each period, powder was taken from the relevant sample and re-dispersed in water. The suspension stability was evaluated by particle-size distribution and content analysis using the protocols previously described.

5.8. Ex Vivo Corneal Drug Penetration Experiment

Porcine eyes were obtained from Lahav Animal Research Institute (Kibbutz Lahav, Israel). The enucleated eyes were kept on ice during transportation and used within 3 hours of enucleation. Corneas surrounded by approximately 5 mm of sclera were dissected and placed on Franz diffusion cells (Permegear Inc., Hellertown, Pa., USA) with an effective diffusion area of 1.0 cm² and a receiver compartment of 8 mL. Dulbecco's phosphate-buffered saline (PBS) (pH=7.0) mixed with 10% ethanol was placed in the receiver chamber maintained at 35° C. and continuously stirred. ³H-Tacrolimus loaded into the NEs/NPs formulations and the control containing ³H-Tacrolimus in castor oil were applied to the mounted cornea. 24 h after the beginning of the experiment, the distribution of radioactivity-labeled ³H-Tacrolimus was determined in the several compartments. First, the remaining formulation on the corneal surface was collected by serial washings with the receptor medium. The cornea was then chemically dissolved with Solvable® in a water bath kept at 60° C. until complete tissue disintegration. Finally, aliquots of the receptor fluid were also collected. Radiolabeled Tacrolimus was determined in Ultima-gold® scintillation liquid in a Tri-Carb 4910 TR beta counter (PerkinElmer, USA).

5.9. Ex Vivo Corneal Toxicity Assessment

5.9.1. MTT Viability Assay

Porcine eyes kept under the same conditions previously described were used for the viability assay. Corneas surrounded by approximately 5 mm of sclera were dissected and disinfected 5 min in 20 mL povidone-iodine solution. Corneas were then washed in PBS and treated with 10 μL of the different concentrations of NCs and incubated at 37° C. in 1.5 mL DMEM for 72 h. To assess the corneal cells viability following the different treatments, MTT viability assay was performed. MTT powder was first dissolved in PBS to prepare a stock solution of 5 mg/mL. This solution was further diluted in PBS to 0.5 mg/mL and 500 μL of the diluted solution were added to each cornea prior to 1 h of incubation. Dye extraction was performed by using 700 μL isopropanol for each cornea and shaking during 30 min at room temperature. Following the latter process, 100 μL of the extract was taken and read in Cytation 3 imaging reader from BioTek at a wavelength of 570 nm.

5.9.2. Epithelial Thickness Measurement

Dissected corneas, treated and incubated according to the same protocol previously described, were immersed in paraformaldehyde for 12 h and further transferred in ethanol until histological sectioning. Samples were cut at 4 μm and stained by Hematoxylin and Eosin. Histology pictures were taken by Olympus B201 microscope (optical magnification of ×40, Olympus America, Inc., MA, USA). Using Image J software, epithelial thickness was obtained by dividing measured epithelial area by its length.

### 6. Results

6.1. Nanoemulsions (NEs)

6.1.1. Composition and Characterization

Numerous NEs were prepared by varying the surfactants and the drug concentrations, the screening aimed to find a physically and chemically stable formulation with submicronic droplets presenting a narrow size distribution. Physico-chemical characteristics of the NEs obtained are summarized in Table 9. Only the formulations containing PVA as a surfactant in the aqueous phase and castor oil in the organic phase were physically stable (NE-5 to NE-8). NE-6 to NE-8 were selected for further evaluation. These NEs differed principally in the concentration of the organic phase surfactant Tween 80 and exhibited a low polydispersity index (PDI) and an average droplet diameter varying from 176 to 201 nm measured with Zetasizer Nano ZS.

TABLE 9
Composition and properties of the different NEs formulations.

| | | | Surfactant in | | | | | | |
|---|---|---|---|---|---|---|---|---|---|
| | | | organic phase | | Surfactant in | | | | |
| | | | Tween | Lipoid | aqueous phase | | Mean | | |
| | Tacrolimus | Oil | 80 | E80 | Solutol | PVA | diam. | | |
| Formul. | W/V %^{a} | Type | W/V %^{a} | W/V %^{a} | W/V %^{a} | W/V %^{a} | (nm) | PDI | Remarks |
| NE-1 | 0.25 | Castor | 0.5 | — | 0.5 | — | 164.3 | 0.11 | aggregates |
| | | MCT | | | | | | | |
| NE-2 | 0.25 | Castor | — | 0.8 | 0.5 | — | 124.1 | 0.12 | aggregates |
| | | MCT | | | | | | | |
| NE-3 | 0.25 | Castor | — | 0.8 | 0.5 | — | 132.6 | 0.13 | aggregates |
| NE-4 | 0.25 | Castor | — | 0.8 | — | 1.4 | 120.2 | 0.1 | aggregates |
| | | MCT | | | | | | | |
| NE-5 | 0.25 | Castor | 3.8 | — | — | 1.4 | 232.3 | 0.24 | — |
| NE-6 | 0.1 | Castor | 1.4 | — | — | 1.4 | 201.2 | 0.15 | — |
| NE-7 | 0.1 | Castor | 0.9 | — | — | 1.4 | 195.8 | 0.10 | — |
| NE-8 | 0.1 | Castor | 0.4 | — | — | 1.4 | 176.7 | 0.11 | — |

^{a}in the formulation after evaporation.

Since the regular Zetasizer Nano ZS is limited for measurements of micronic particles, a confirmation of particle size distribution for the NEs' droplets can be made by means of laser diffractometry using a Mastersizer 2000 (Malvern Instruments, UK), covering a size range of 0.02-2000 μm. As it can be seen in FIG. 5 obtained by the instrument, the selected formulations (NE-6 to NE-8) exhibited a submicronic profile that was similar for all the NEs tested, confirming the results obtained by the Zetasizer Nano ZS.

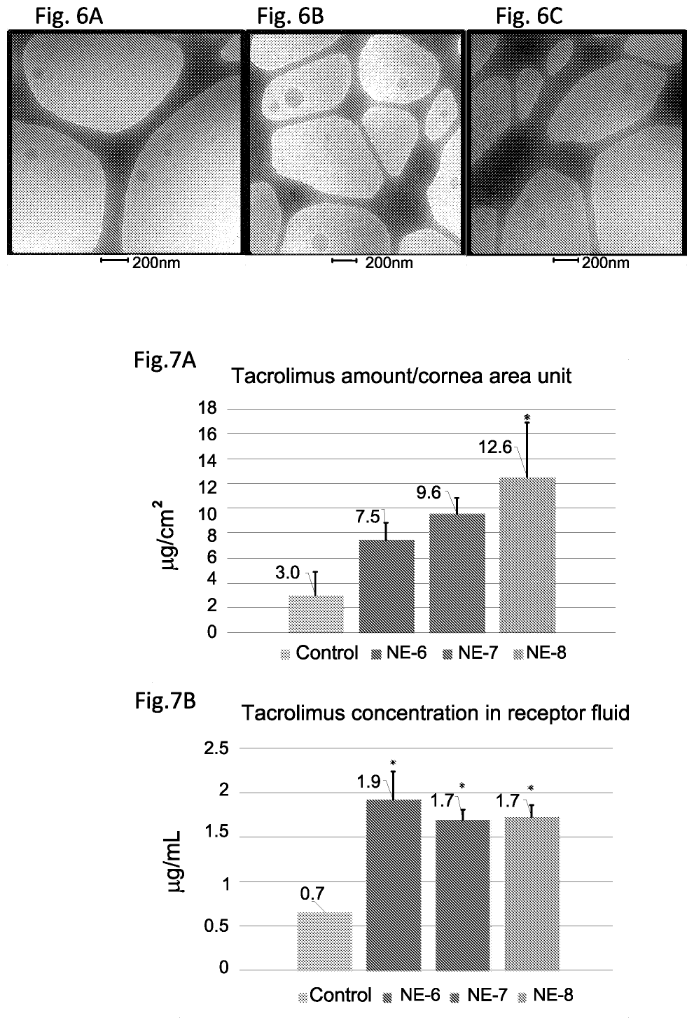

Morphological examination of the selected NEs was carried out to complete their physicochemical characterization. Spherically-shaped NEs droplets were observed in all the formulations (FIG. 6).

### 6.1.2. Ex Vivo Corneal Penetration Experiment

The results reported in FIG. 7 exhibit the amount of [³H]-Tacrolimus in the cornea per area unit (FIG. 7A) and its concentration in the receptor compartment (FIG. 7B) following topical application of [³H]-Tacrolimus-loaded NEs and the oil control after 24 h. All the tested NEs were diluted to obtain a Tacrolimus concentration of 0.05% and were adjusted to isotonicity.

Tacrolimus loaded in NE-8 was significantly more retained in the cornea compared to the oil control (p<0.05). The drug concentration in the receptor fluid was also four fold higher in NE-6, 7 and NE-8 compared to the control (p<0.05) highlighting the significant increase in Tacrolimus penetration through the cornea when loaded in nanoemulsions. However, between the NEs tested, no difference in permeation was found (p>0.05).

### 6.1.3. Stability Assessment

The three selected NEs displayed conserved physico-chemical characteristics and drug content after eight weeks when stored at 4° C. and room temperature. However, at 37° C., after the same period, tacrolimus content (in w/v) decreased by a minimum of 20% from the initial drug content as it can be seen in Table 10.

TABLE 10
Stability results of the selected NEs after eight
weeks at different storage temperatures.

| | 4° C. | | | 37° C. | | | Room temperature | | |
|---|---|---|---|---|---|---|---|---|---|
| | Size | | Content | Size | | Content | Size | | Content |
| | (nm) | PDI | (%) | (nm) | PDI | (%) | (nm) | PDI | (%) |
| Initially | 201.2 | 0.15 | 0.06 | 201.2 | 0.15 | 0.06 | 201.2 | 0.15 | 0.06 |
| 8 weeks | 200.3 | 0.11 | 0.04 | 200.3 | 0.11 | 0.04 | 201.1 | 0.14 | 0.05 |
| Initially | 195.8 | 0.1 | 0.05 | 195.8 | 0.1 | 0.05 | 195.8 | 0.1 | 0.05 |
| 8 weeks | 195.3 | 0.12 | 0.05 | 194.8 | 0.09 | 0.04 | 195.1 | 0.1 | 0.05 |
| Initially | 176.7 | 0.11 | 0.05 | 176.7 | 0.11 | 0.05 | 176.7 | 0.11 | 0.05 |
| 8 weeks | 180.3 | 0.11 | 0.05 | 178.7 | 0.11 | 0.04 | 177.1 | 0.11 | 0.05 |

### 6.2. Nanoparticles

Numerous nanoparticles' formulations were prepared by varying PLGA MW, oil, surfactants, drug and their concentrations, and preparing either Nanocapsules (NCs) or Nanospheres (NSs). This screening aimed to find a stable formulation with particles presenting a narrow size distribution and a high encapsulation efficiency.

6.2.1. Nanospheres (NSs)

All the attempts to formulate tacrolimus in NSs were unsuccessful, after a few hours, aggregates formed (Table 11). Oil to dissolve Tacrolimus seemed to be essential to formulate the drug and obtain a stable product.

TABLE 11
Composition of the different NSs formulations.

| | | | Surfactant in | | Surfactant in | |
|---|---|---|---|---|---|---|
| | PLGA | | organic phase | | aqueous phase | |
| | W | | Tween 80 | Lipoid E80 | Solutol | |
| Formulation | kDa) | (W/V %)^{a} | (W/V %)^{a} | (W/V %)^{a} | (W/V %)^{a} | Remarks |
| NS-1 | 100 | 0.7 | 0.1 | — | 0.5 | aggregates |
| NS-2 | 00 | 0.7 | 0.1 | — | 0.5 | aggregates |
| NS-3 | 100 | 0.7 | 0.5 | — | 0.5 | aggregates |
| NS-4 | 60 | 0.7 | 0.5 | — | 0.5 | aggregates |
| NS-5 | 50 | 0.7 | — | 0.5 | 0.5 | aggregates |

^{a}in the formulation after evaporation

### 6.2.2. Nanocapsules (NCs)

### 6.2.2.1. Composition and Characterization

Based on the physical stability of the NEs when formulated with castor oil as the only oil type, we formulated the NCs with the same component. Various parameters in the formulations were changed such as the PLGA molecular weight and the concentration and type of surfactants used in aqueous and organic phase (Table 12).

TABLE 12
Composition of the different NCs formulations.

| | | | | | Surfactant in organic phase | | | Surfactant in | |
|---|---|---|---|---|---|---|---|---|---|
| | PLGA | | | | Tween | Cremophor | Lipoid | aqueous phase | |
| | W | | Tacrolimus | Castor oil | 80 | EL | E80 | Solutol | PVA |
| Formulation | (kDa) | (W/V %)^{a} | (W/V %)^{a} | (W/V %)^{a} | (W/V %)^{a} | (W/V %)^{a} | (W/V %)^{a} | (W/V %)^{a} | (W/V %)^{a} |
| NC-1 | 50 | 0.6 | 0.05 | 1.05 | — | 0.25 | — | — | 1.4 |
| NC-2 | 50 | 0.6 | 0.05 | 1.05 | 0.2 | — | — | 0.2 | — |
| NC-3 | 50 | 0.6 | 0.05 | 1 | 0.4 | — | — | 0.2 | — |
| NC-4 | 50 | 0.6 | 0.05 | 1 | 0.3 | — | — | — | 1.4 |
| NC-5 | 50 | 0.6 | 0.1 | 1 | 0.2 | — | — | 0.2 | — |
| NC-6 | 50 | 0.6 | 0.05 | 1.1 | — | 0.25 | — | 0.2 | — |
| NC-7 | 50 | 0.6 | 0.05 | 1.1 | — | 0.5 | — | 0.2 | — |
| NC-8 | 50 | 0.6 | 0.05 | 0.9 | — | — | 0.3 | 0.5 | — |
| NC-9 | 50 | 0.6 | 0.07 | 0.9 | — | — | 0.3 | 0.5 | — |
| NC-10 | 50 | 0.6 | 0.1 | 0.9 | — | — | 0.3 | 0.5 | — |
| NC-11 | 50 | 0.6 | 0.1 | 1 | — | — | 0.3 | 0.2 | — |
| NC-12 | 50 | 0.6 | 0.1 | 0.9 | — | — | 0.5 | 0.5 | — |
| NC-13 | 50 | 0.6 | 0.1 | 1.2 | — | — | 0.3 | 0.5 | — |
| NC-14 | 50 | 0.6 | 0.1 | 0.9 | — | — | 0.3 | 0.25 | — |
| NC-15 | 4.5 | 0.6 | 0.1 | 0.9 | — | — | 0.3 | 0.5 | — |
| NC-16 | 7.5 | 0.6 | 0.1 | 0.9 | — | — | 0.3 | 0.5 | — |
| NC-17 | 17 | 0.6 | 0.1 | 0.9 | — | — | 0.3 | 0.5 | — |
| NC-18 | 100 | 0.6 | 0.1 | 0.9 | — | — | 0.3 | 0.5 | — |
| NC-19 | 100 | 0.6 | 0.1 | 1.2 | — | — | 0.3 | 0.5 | — |

The most stable formulations were selected for further characterization (Table 13). Except for NC-18 formulated with PLGA 100 KDa, all the NCs were formulated with PLGA 50 KDa. NCs' size varied from 90 to 165 nm and presented a PDI below or equal to 0.1, highlighting the homogeneity of the NCs formed. The encapsulation efficiencies (EEs) obtained did not differ much when changing the different parameters and reached a maximum of 81%.

TABLE 13
Properties of the selected NCs formulations.

| Formulation | Mean diameter (nm) | PDI | EE (%) |
|---|---|---|---|
| NC-1 | 165.7 | 0.08 | 79 |
| NC-2 | 165.1 | 0.1 | 79 |
| NC-5 | 162.8 | 0.1 | 77 |
| NC-6 | 155.9 | 0.08 | 81 |
| NC-10 | 106.5 | 0.09 | 61 |
| NC-18 | 90.8 | 0.08 | 73 |

### 6.2.2.2. Lyophilization

Because of the PLGA NCs' instability in aqueous medium, lyophilization was performed. Screening of cryoprotectants at variable ratios was achieved in order to identify the most efficient compound able to prevent particles aggregation. Concentration of these compounds in the final reconstituted product was taken into account in the ratios tested to fill FDA requirements. Sucrose and trehalose were found to be inadequate for NCs lyophilization owing to a lack of cake at ratios PLGA:Cryoprotectants varying from 1:1 to 1:20. Mannitol gave a cake, however, after reconstitution, aggregates were seen at ratios from 1:1 to 1:6 (Table 14).

TABLE 14
Appearance, particle size and PDI value of the selected
NCs using various cryoprotectants with different ratios.

| | Ratio | Before | After | Mean | |
|---|---|---|---|---|---|
| Cryo- | PLGA:Cryo- | reconsti- | reconsti- | diameter | |
| protectant | protectant | tution | tution | (nm) | PDI |
| Sucrose/ | 1:1 | No cake | — | — | — |
| Trehalose | 1:2 | No cake | — | — | — |
| | 1:5 | No cake | — | — | — |
| | 1:10 | No cake | — | — | — |
| | 1:15 | No cake | — | — | — |
| | 1:20 | No cake | — | — | — |
| Mannitol | 1:1 | Good cake | Aggregates | — | — |
| | 1:2 | Good cake | Aggregates | — | — |
| | 1:4 | Good cake | Aggregates | — | — |
| | 1:6 | Good cake | Aggregates | — | — |

For the selected NCs, β-Cyclodextrin was the only cryoprotectant that gave a good cake and a quick redispersion in water. Regarding size similarity before and after the process, along with a relatively low PDI, best lyophilization results were obtained for NC-1 and NC-2 formulations. The preferred ratio PLGA: β-Cyclodextrin was 1:10 for both NCs (Table 15).

TABLE 15
Appearance, particle size and PDI value of NC-1 and
NC-2 using different ratios of β-Cyclodextrin.

| | Ratio | Before | After | Mean | |
|---|---|---|---|---|---|
| Formu- | PLGA:β- | reconsti- | reconsti- | diameter | |
| lation | Cyclodextrin | tution | tution | (nm) | PDI |
| NC-1 | 1:1 | Good cake | Big aggregates | — | — |
| | 1:3 | Good cake | Big aggregates | — | — |
| | 1:5 | Good cake | Big aggregates | — | — |
| | 1:7 | Good cake | Good | 233.7 | 0.37 |
| | 1:8 | Good cake | Good | 225.9 | 0.25 |
| | 1:10 | Good cake | Good | 165.4 | 0.18 |
| NC-2 | 1:1 | Good cake | Small grains | 250.2 | 0.24 |
| | 1:3 | Good cake | Small grains | 225.3 | 0.19 |
| | 1:6 | Good cake | Good | 200.4 | 0.19 |
| | 1:8 | Good cake | Good | 190.3 | 0.17 |
| | 1:10 | Good cake | Good | 170.2 | 0.15 |

Consequently, the lead formulations were NC-1 and NC-2, differing in the surfactants used in aqueous and organic phases. NC-1 contained Cremophor EL and PVA whereas NC-2 was formulated with Tween 80 and Solutol. These two NCs formulations preserved their initial size of approximately 170 nm, with a low PDI and an encapsulation efficiency of 70% after lyophilization process as it can be seen in Table 16.

TABLE 16
Lead NCs properties before and after lyophilization

| | | |
|---|---|---|
| Formulation | NC-1 | NC-2 |

Before Lyophilization

| | | |
|---|---|---|
| Size (nm) | 165.7 | 165.1 |
| PDI | 0.08 | 0.1 |
| EE(%) | 81 | 79 |

After Lyophilization

| | | |
|---|---|---|
| Size (nm) | 165.4 | 170.2 |
| PDI | 0.18 | 0.15 |
| EE(%) | 70 | 71 |

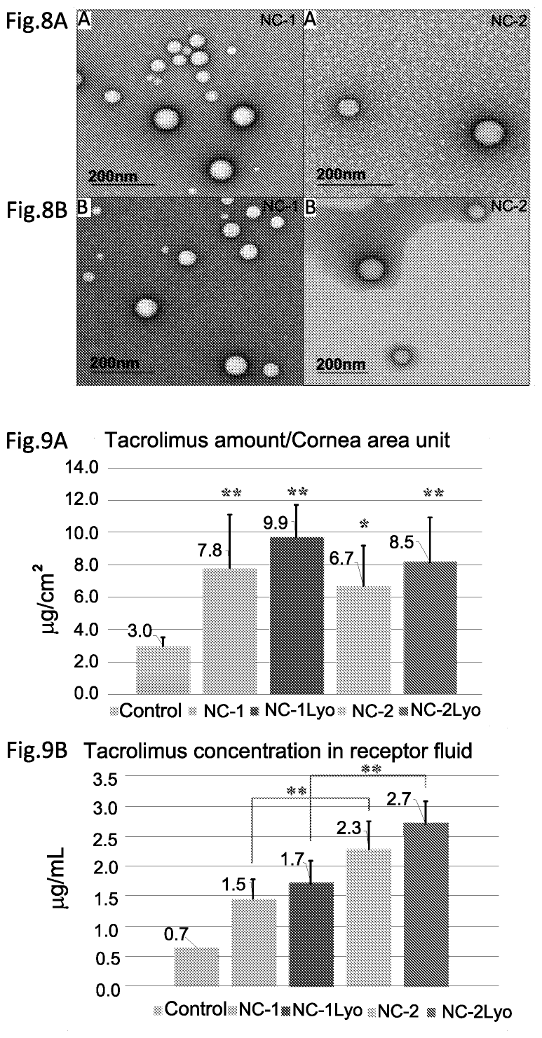

Morphological examination was also assessed by TEM (FIG. 8). The two formulations evaluated presented spherical-shaped NCs before lyophilization (FIG. 8A). Lyophilization and powder reconstitution in water did not affect the particles' physical aspect and no aggregation was seen (FIG. 8B).

### 6.2.2.3. Ex Vivo Corneal Penetration Experiment

Aiming to assess the potential of tacrolimus to permeate the cornea when loaded in NCs, penetration experiment of radiolabeled formulations was performed. The results reported in FIG. 9 exhibit the amount of [³H]-Tacrolimus in the cornea per area unit (FIG. 9A) and its concentration in the receptor compartment (FIG. 9B) following topical application of [³H]-Tacrolimus-loaded NCs and the oil control after 24 h. The two NCs formulations were tested before and after lyophilization and reconstitution in water to obtain a Tacrolimus concentration of 0.05% w/v.

All the NCs treatments significantly retained more Tacrolimus in the cornea compared to the oil control (*p<0.05, **p<0.01). The same result was obtained for the drug concentration in the receptor fluid which was significantly higher in comparison to control (**p<0.01). Moreover, these results showed the better drug permeation through the cornea when loaded in NC-2 compared to NC-1 (**p<0.01), highlighting the importance of the surfactants used in the formulations. No differences were seen in these observations after lyophilization and aqueous reconstitution (p>0.05) suggesting that this process did not alter NCs' properties.

### 6.2.2.4. Stability Assessment

The two selected NCs formulations displayed a different stability profile when stored over time at different temperatures. After eight weeks, at 37° C., NC-1's size and PDI increased and initial drug content (w/w) decreased by approximately 20% (Table 17). On the contrary, NC-2 conserved its physico-chemical characteristics and initial drug content during the storage time tested (Table 18). These results suggested that the choice of surfactants in formulations is also critical to keep initial NCs' properties over time.

TABLE 17
NC-1 stability results over time at different storage temperatures

| | 4° C. | | | Room temperature | | | 37° C. | | |
|---|---|---|---|---|---|---|---|---|---|
| | Size | | Content | Size | | Content | Size | | Content |
| | (nm) | PDI | (% w/w) | (nm) | PDI | (% w/w) | (nm) | PDI | (% w/w) |
| Initially | 165.4 | 0.18 | 0.6 | 165.4 | 0.18 | 0.6 | 165.4 | 0.18 | 0.6 |
| 1 week | 165 | 0.19 | 0.6 | 164.1 | 0.18 | 0.6 | 167.2 | 0.19 | 0.6 |
| 2 weeks | 164 | 0.19 | 0.6 | 164.3 | 0.19 | 0.6 | 169.3 | 0.19 | 0.6 |
| 4 weeks | 170 | 0.20 | 0.6 | 161.7 | 0.22 | 0.6 | 173.2 | 0.23 | 0.6 |
| 8 weeks | 165.9 | 0.22 | 0.6 | 165.3 | 0.23 | 0.6 | 178.3 | 0.23 | 0.5 |

TABLE 18
NC-2 stability results over time at different storage temperatures

| | 4° C. | | Room temperature | | | 37° C. | | | |
|---|---|---|---|---|---|---|---|---|---|
| | Size | | Content | Size | | Content | Size | | Content |
| | (nm) | PDI | (% w/w) | (nm) | PDI | (% w/w) | (nm) | PDI | (% w/w) |
| Initially | 170.2 | 0.15 | 0.5 | 170.2 | 0.15 | 0.5 | 170.2 | 0.15 | 0.5 |
| 2 weeks | 169.1 | 0.11 | 0.5 | 170.1 | 0.14 | 0.5 | 169.3 | 0.13 | 0.5 |
| 4 weeks | 169.9 | 0.11 | 0.5 | 170.8 | 0.12 | 0.5 | 170 | 0.12 | 0.5 |
| 8 weeks | 169.2 | 0.12 | 0.5 | 172.2 | 0.13 | 0.5 | 171.7 | 0.14 | 0.5 |
| 12 weeks | 167.6 | 0.12 | 0.5 | 172.7 | 0.12 | 0.5 | 173.3 | 0.13 | 0.5 |
| 17 weeks | 178.7 | 0.13 | 0.5 | 171.6 | 0.13 | 0.5 | 178.3 | 0.13 | 0.5 |

6.2.3 Comparison of NCs Vs NEs Ex Vivo Corneal Penetration

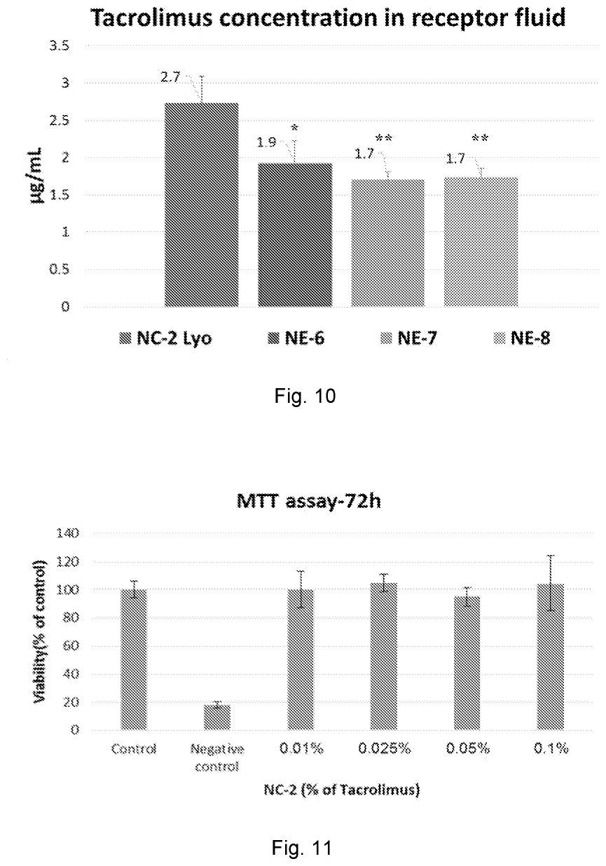

In order to evaluate the potential superiority of one of the tacrolimus loaded nanocarriers over the second one regarding the cornea penetration, comparison of the results obtained was performed. Statistical analysis suggested that fresh NCs along with lyophilized NC-1, did not penetrate more the cornea compared to NEs (p>0.05). However, lyophilized NC-2 delivered, through the cornea, a higher tacrolimus amount than the different NEs (*p<0.05, **p<0.01) as it can be seen in FIG. 10.

### 6.2.4. Ex Vivo Toxicity Assessment

### 6.2.4.1. MTT Viability Assay

As a result of cornea penetration experiment success and its conserved stability over time, NC-2 became the lead formulation. In order to evaluate its toxicity on corneal cells, different concentrations of isotonic, reconstituted NC-2 were tested on ex vivo pig corneas incubated during 72 h in organ culture. MTT assay performed afterwards, suggested that the NCs did not affect the viability of the tissues at the concentrations evaluated compared to the control untreated corneas (p>0.05) as shown in FIG. 11.

### 6.2.4.2. Epithelial Thickness Measurement

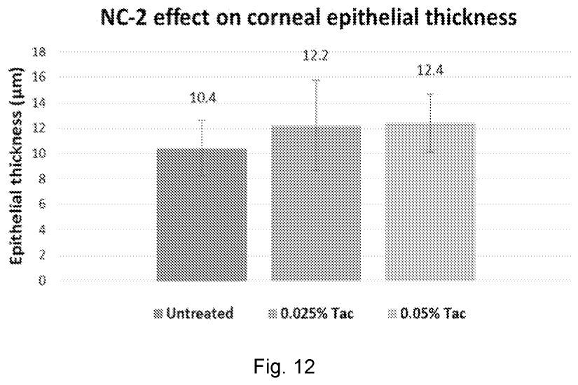

In the objective to assess a potential harm of the corneal epithelium provoked by NC-2 application, histology and H&E staining of the treated ex vivo pig corneas were performed after 72 h incubation followed by epithelial thickness measurement. The results obtained exhibited similar epithelial thickness between NC-2 treated corneas and the untreated control (p>0.05) suggesting that the tested NCs' concentrations did not affect the cornea morphology (FIG. 12).

### 7. Discussion

The design of an immunosuppressant drug delivery system targeting the eye first required the development of nanocarriers which would encapsulate the immunosuppressant, and would have the potential to penetrate efficiently the highly selective cornea barrier of the eye.

In the present research, the immunosuppressant Tacrolimus was encapsulated within biodegradable PLGA-based nano-particulate delivery system or loaded in oil in water nanoemulsions. The solvent displacement method, a popular and suitable technique for lipophilic drug encapsulation, was adopted in this study for the preparation of both NEs, NSs and NCs, with different surfactants, PLGA MWs, tacrolimus and oil concentrations. Only NEs formulations containing PVA as a surfactant in the aqueous phase were physically stable probably because of the ability of the acetate groups of the polymer to adsorb to the hydrophobic surface of the oil droplets along with the strong solvation (hydration) of the stabilizing chain, resulting in an effective steric hindrance. Moreover, polymeric surfactants such as PVA increase the viscosity of the aqueous phase which maintain the nanodroplets in suspension. The NEs formulations selected, varying in the organic phase surfactant (Tween 80) concentration, presented all the desired physicochemical properties. Indeed, nanodroplets exhibited a mean size varying from 176 to 201 nm, a low polydispersity index (˜0.1) and physical stability. After the tacrolimus NEs were characterized and optimized, their cornea penetration/permeation profile was evaluated by using Franz diffusion cells. The distribution of [³H]-Tacrolimus from both NEs and the oil control was determined in the different compartments. The results revealed that the penetration of [³H]-Tacrolimus through the cornea was more than two-fold greater than for the oil control (FIG. 7B).

This finding is particularly important because tacrolimus has difficulty penetrating the corneal epithelium and accumulates in the corneal stroma due to its poor water solubility and relatively high molecular weight, however, when loaded in the nanoemulsions, tacrolimus more permeated to the cell receptor fluid suggesting that the drug penetrated both the lipophilic and hydrophilic parts composing the complex cornea tissue.

These results correspond to those from previous reports in the literature, showing that the use of a nanoemulsion carrier can improve the penetration of drugs through the cornea owing to the uptake of the colloidal droplets by the corneal epithelium.

From these Franz cell experiment results, it should also be emphasized that there was no significant decrease in cornea penetration when decreasing Tween80 concentration from 1.4% in NE-6 to 0.4% in NE-8, suggesting that a minimal amount of this surfactant can be used without affecting its potential to act as a penetration enhancer.

Physico-chemical stability evaluation performed in accelerated temperature conditions, of the three selected NEs (NE-6 to NE-8) showed that although the physical stability of the NEs was conserved with a similar size and PDI of the droplets in all the temperatures tested, at 37° C., the drug content decreased after eight weeks to 80% of the initial tacrolimus concentration. These findings suggest that in view of the partition of the drug between the oil and aqueous phases, tacrolimus was probably degraded as a result of the water presence.

Therefore, to overcome the instability of the NEs formulations in aqueous medium, it was decided to concentrate all the efforts on the optimization of a NP formulation which will also be subjected to lyophilization and reconstitution prior to use. Attempts to encapsulate the highly lipophilic Tacrolimus into NSs were unsuccessful. Indeed, after a few minutes, the drug aggregated. The instability of this nanocarrier can have multiple reasons. First, tacrolimus may have higher affinity to the surfactants than to the PLGA polymer, causing the micellization of the drug instead of its encapsulation. Moreover, tacrolimus may adsorb to the polymer surface resulting in drug aggregation at equilibrium when the drug passes to the aqueous phase.

In addition, the small size of the NSs increases the free energy of Gibbs, therefore, the particles tend to assemble themselves to decrease the surface energy provoking their collision, the release of the drug and its crystallization. Designing NCs seemed to be a better solution to encapsulate Tacrolimus because of the oil component that will dissolve the drug. Screening of many formulations was achieved by changing the NCs' components and their concentrations. The selected NCs exhibited a mean size under 170 nm, a low PDI (≤0.1) and encapsulation efficiencies varying from 61% for NC-10 to 81% for NC-6. Therefore, the next step required was to perform lyophilization of the NCs in order to prevent both tacrolimus and PLGA degradation in aqueous environment.

An adequate lyophilization method would have three required criteria: an intact cake occupying the same volume as the original frozen mass; the reconstituted NCs would have a homogeneous suspension appearance without aggregates; and finally, upon water reconstitution, the NCs' initial physicochemical properties should be maintained. Numerous parameters affect the resistance of NCs to the stress imposed by lyophilization, including the type and concentration of the cryoprotectant. In order to choose the appropriate cryoprotectant, a screening of many of them at variable concentrations was performed. For all the selected NCs, different ratios of sucrose and trehalose did not give conserved cakes. In spite of intact cakes that were obtained after using mannitol as cryoprotectant, aqueous reconstitution was not homogeneous. However, with β-cyclodextrin, at a ratio of 1:10, lyophilization was optimal with both conserved cake, homogeneous aqueous reconstitution and no alteration in physico-chemical characteristics for two out of the six selected NCs. NC-1 and NC-2, differing in the surfactants used in aqueous and organic phases, became the lead formulations for the next experiments. Morphological examination revealed high resemblance before and after lyophilization for the two formulations, with conserved spherical shape of the particles and no aggregation noticed. These two formulations were further tested on Franz cells to evaluate their potential for corneal retention and penetration. The distribution of [³H]-Tacrolimus from NC-1, NC-2, their respective lyophilized powders and the oil control was determined in the different compartments. The results first revealed that there was no difference between fresh formulations and lyophilized ones neither in cornea retention nor in its penetration, suggesting that this process did not alter NCs' properties. Second, [³H]-Tacrolimus was more than two-fold more retained in the cornea when in NCs than the oil control (FIG. 9A). Moreover, the drug concentration was up to four fold higher in the receptor than the oil control (FIG. 9B). Third, it is also important to emphasize the significant difference in [³H]-Tacrolimus concentration in the receptor fluid between NC-1 and NC-2. These formulations differing in the surfactants composing them were tested to assess the influence of these compounds on penetration enhancement. NC-2 that contained Tween 80 in the organic phase and Solutol in the aqueous phase exhibited a better cornea penetration than NC-1 containing Cremophor EL in the organic phase and PVA in the aqueous phase. Being both polyoxyethylated nonionic surfactants, Tween80 and Cremophor EL were assumed not to be involved in these differences. On the opposite, PVA used in the aqueous phase is a polymeric surfactant having a different mechanism of action, which consists in steric hindrance as it has been said previously. In addition, in the formulation of PLGA nanoparticles, the hydrophobic fraction of PVA forms a network on the polymer surface altering the surface hydrophobicity of the particles. Moreover, it has been reported that this alteration can affect the cellular uptake of these particles, a mechanism involved in ocular penetration. Therefore, the decreased penetration of NC-2 formulated with PVA may be due to a reduction in corneal epithelium uptake occurring when colloidal drug delivery systems are applied topically to the eye. Comparison of NEs and NCs suggested that both nanocarriers were superior to the control to achieve drug penetration through cornea, but no significant differences were found between fresh NCs and NEs as it has already been reported. Nevertheless, cornea penetration of lyophilized NC-2 was significantly superior to NEs. This result is in contradiction with studies previously published showing that there were no differences between corneal penetration of colloidal nanocarriers and that lyophilization of the particles with B-Cyclodextrin decreased the ocular permeation. Our results might be due to a better encapsulation of the drug leading to less complex formation between nonentrapped tacrolimus and the ß-Cyclodextrin which results in increased drug penetration by means of nanocapsules' uptake, a process not occurring when the free drug is complexed with the cryoprotectant. Stability assessment of the lyophilized selected NCs showed that only in NC-2 the initial drug content was conserved over time in accelerated conditions. On the contrary, NC-1 tacrolimus content decreased by 17% after eight weeks in 37° C., probably because of the effects some surfactants can have on accelerating drug degradation. In view of the better penetration and stability results achieved by NC-2, it became the lead formulation for the future experiments. NC-2 toxicity on corneal epithelium was assessed both by MTT experiment and histological measurement. The lyophilized powder reconstituted with water to obtain different drug concentrations proved to conserve the viability of corneal cells and to preserve the corneal epithelium integrity, suggesting that topical eye instillation of this formulation may be safe for patients.

### 8. Dexamethasone Palmitate

8.1 Solubility in FDA Approved Oils for Ophthalmic Use

Dexamethasone palmitate solubility was assessed in mineral oil, castor oil and MCT.

TABLE 19
Dexamethasone assessed in various oils

| | Concentration(mg/mL) |
|---|---|
| Mineral oil | 1.3 |
| Castor oil | 33.6 |
| MCT | 46.6 |

As the highest solubility of the drug was obtained in MCT oil, this oil was chosen for formulation development.

8.2 Nanocarriers Development

Nanoemulsions, nanospheres and nanocapsules were tested in order to choose the most adapted nanocarrier for dexamethasone palmitate. The most important parameters were size, PDI, encapsulation efficiency for nanoparticles and physical stability. The second goals were to obtain a high drug concentration and lyophilization feasibility.

TABLE 20
Nanocarrier development.

| | D 1 | D 2 | D 3 | D 4 | D 5 | D 6 | D 7 | D 8 |
|---|---|---|---|---|---|---|---|---|
| Ingredients (mg) | | | | | | | | |
| DexP. | 16.42 | 16.09 | 40.45 | 40.47 | 39.99 | 40 | 39.99 | 40.01 |
| PLGA (0.15-0.25 g/dL) | 60.43 | 60.32 | 0 | 0 | 60.29 | 0 | 60.18 | 60.31 |
| PLGA 17k Purac | 0 | 0 | 0 | 0 | 0 | 60.39 | 0 | 0 |
| Tween 80 | 20.54 | 25.5 | 52.95 | 53.01 | 26.89 | 27.89 | 24.51 | 0 |
| TYLOXAPOL | 0 | 0 | 0 | 0 | 0 | 0 | 0 | 17.49 |
| Castor oil | 0 | 0 | 0 | 41.2 | 0 | 0 | 0 | 0 |
| MCT | 0 | 25.96 | 45.1 | 0 | 52.35 | 50.27 | 100.1 | 51.03 |
| Acetone (mL) | 10 | 10 | 10 | 10 | 10 | 10 | 10 | 10 |
| Solutol | 20 | 20 | 20 | 20 | 20 | 20 | 20 | 20 |
| Kolliphor RH 40 | 0 | 0 | 0 | 0 | 0 | 0 | 0 | 0 |
| Water (mL) | 20 | 20 | 20 | 20 | 20 | 20 | 20 | 20 |
| Final volume (mL) | 10 | 10 | 6 | 5 | 10 | 10 | 10 | 10 |
| Concentration (mg/mL) | 1.684 | 1.911 | 7.611 | 8.968 | N/A | 4.23 | N/A | 4.31 |
| EE(%) | 58.6 | 82 | N/A | N/A | N/A | 91 | N/A | 98 |
| Fresh formulations | | | | | | | | |
| Size (nm) | 99.49 | 121.1 | 108.4 | 113.9 | 149 | 127.2 | 167.9 | 156.4 |
| PDI | 0.093 | 0.078 | 0.102 | 0.084 | 0.103 | 0.069 | 0.103 | 0.102 |
| | D 9 | D 10 | D 11 | D 12 | D 13 | D 14 | D 15 | D 16 |
| Ingredients (mg) | | | | | | | | |
| DexP. | 30.49 | 30.05 | 40.02 | 40.12 | 60 | 60.14 | 40.02 | 40.06 |
| PLGA (0.15-0.25 g/dL) | 0 | 0 | 0 | 0 | 0 | 0 | 0 | 59.98 |
| PLGA 17k Purac | 0 | 0 | 60.14 | 0 | 60.34 | 60.02 | 60 | 0 |
| Tween 80 | 0 | 48.39 | 0 | 0 | 30.66 | 0 | 25 | 0 |
| TYLOXAPOL | 0 | 0 | 11.44 | 16.14 | 0 | 12.46 | 0 | 14.46 |
| Castor oil | 0 | 0 | 0 | 0 | 0 | 0 | 0 | 0 |
| MCT | 0 | 0 | 0 | 50.45 | 50.35 | 50.03 | 49.54 | 49.86 |
| Acetone (mL) | 10 | 10 | 10 | 10 | 10 | 10 | 10 | 10 |
| Solutol | 0 | 20 | 20 | 20 | 20 | 20 | 0 | 0 |
| Kolliphor RH 40 | 0 | 0 | 0 | 0 | 0 | 0 | 50.2 | 50.2 |
| Water (mL) | 20 | 20 | 20 | 20 | 20 | 20 | 20 | 20 |
| Final volume (mL) | 10 | 10 | 10 | 5 | 10 | 10 | 10 | 10.12 |
| Concentration (mg/mL) | 3.05 | 3.005 | 4.1 | 8 | 6.35 | 6.15 | 4.26 | 4.1 |
| EE(%) | N/A | N/A | N/A | N/A | 92.5 | 96 | 84 | 92 |
| Fresh formulations | | | | | | | | |
| Size (nm) | 144.8 | 66.78 | 111.8 | 153.4 | 140.2 | 151.9 | 126.2 | 140.9 |
| PDI | 0.055 | 0.08 | 0.083 | 0.078 | 0.058 | 0.062 | 0.067 | 0.082 |

8.3 Lyophilization with HydroxyPropyl-β-Cyclodextrin at Different Ratios with PLGA was Performed.

As shown in Table 21, empty boxes mean that the powder reconstitution with water was not homogeneous. Grey boxes represent the best physical parameters obtained with the minimum ratio of cryoprotectant.

TABLE 21
Lyophilization of nanoemulsion.

| | | D | D | D | D | D | D | D | D | D | D | D | D | D | D |
|---|---|---|---|---|---|---|---|---|---|---|---|---|---|---|---|
| PLGA:HPBCD | | 1 | 2 | 3 | 4 | 5 | 6 | 7 | 8 | 11 | 12 | 13 | 14 | 15 | 16 |
| 1:2 | Size | | | 95.34* | | | | | | | | | | | |
| | PDI | | | 0.123 | | | | | | | | | | | |
| 1:5 | Size | | | 140.6 | | | | | | | 333.9 | | 333.9 | | |
| | PDI | | | 0.133 | | | | | | | 0.418 | | 0.418 | | |
| 1:7 | Size | | | | 293.2 | | | | | | | | | | |
| | PDI | | | | 0.244 | | | | | | | | | | |
| 1:10 | Size | | | | 251.2 | | 159.1 | | 172.6 | | 184.6 | 191.1 | 180.8 | 139.8 | 142.4 |
| | PDI | | | | 0.197 | | 0.103 | | 0.119 | | 0.197 | 0.172 | 0.122 | 0.093 | 0.11 |
| 1:12 | Size | | 138 | | 208.2 | 217.3 | 150.7 | | 172.2 | | 176.1 | 177.7 | 172.7 | | |
| | PDI | | 0.092 | | 0.139 | 0.233 | 0.091 | | 0.103 | | 0.175 | 0.111 | 0.093 | | |
| 1:15 | Size | | | | 180.8 | 194.4 | 147 | 214.8 | 168.8 | | 169.1 | 172.8 | 171.8 | | |
| | PDI | | | | 0.106 | 0.158 | 0.049 | 0.226 | 0.099 | | 0.116 | 0.136 | 0.083 | | |

*These lyophilization process results were not reproducible.

8.4 Nanospheres

After a few days, aggregates were seen in nanospheres (D11). Moreover, lyophilization did not work at all the ratios tested. It was therefore decided to continue with nanoemulsions and nanocapsules.

8.5 Nanoemulsions

In order to investigate the importance of the components in nanoemulsions' physical stability, samples D9 and D10 were formulated without oil and/or the different surfactants. Both presented phase separation after a few days.

Samples D3, D4 and D12 succeeded however, D3 was lyophilized at the minimal cryoprotectant concentration but was not reproducible. Nevertheless, for the purpose of comparison with lyophilized nanocapsules the latter was then chosen for further investigation.

8.6 Nanocapsules

The highest drug concentration and encapsulation efficiencies were obtained for D6, D8 and D13 to D16. Lyophilization was also successful at PLGA; HPBCD ratios from 1:10 to 1:15.

8.7 Stability

TABLE 22
Stability of a nanoemulsion -not lyophilized

| | Initially | | | | | | |
|---|---|---|---|---|---|---|---|
| | Storage | | | | | | |
| | Temp. | 3 weeks | | | 6 weeks | | |
| D3 | ° C. | 4 | 25 | 40 | 4 | 25 | 40 |
| Size | 114.7 | 116.2 | 115.5 | 114.9 | 197.8 | 119 | 114.7 |
| PdI | 0.092 | 0.088 | 0.093 | 0.094 | 0.513 | 0.2 | 0.08 |
| Content | 100 | 98 | 98 | 102 | 107 | 105 | 96 |
| (%) | | | | | | | |

As shown in Table 22, after 6 weeks, the size and PDI of the droplets was altered especially at 4 and 25° C. storage Temp., meaning that the nanoemulsion was not stable. A significant increase in the PDI value clearly indicates that the droplet size population is not more homogeneous and the increase in PDI suggest a marked coalescence of oil droplets increasing the diameter size of many oil droplets. This process is irreversible.

Samples D6 and D8 are sample candidates as both showed only a slight size change were seen after 12 weeks.

TABLE 23
Stability of nanocapsules-lyophilized and reconstituted

| | Initially | | | | | | | | | | | | |
|---|---|---|---|---|---|---|---|---|---|---|---|---|---|
| | Storage | | | | | | | | | | | | |
| | Temp. | 2 weeks | | | 4 weeks | | | 8 weeks | | | 12 weeks | | |
| | ° C. | 4 | 25 | 40 | 4 | 25 | 40 | 4 | 25 | 40 | 4 | 25 | 40 |
| D6 | | | | | | | | | | | | | |
| Size | 153.9 | 155.3 | 153.4 | 156.9 | 160.2 | 155.8 | 161.4 | 160.1 | 160.8 | 172.3 | 165.7 | 161.9 | 177.1 |
| PdI | 0.058 | 0.053 | 0.068 | 0.082 | 0.057 | 0.069 | 0.078 | 0.085 | 0.113 | 0.094 | 0.079 | 0.077 | 0.085 |
| Content (%) | 5.34 | 100 | 99 | 99 | 98 | 97 | 99 | 96 | 97 | 96 | 96 | 92 | 93 |
| W. content (%) | 5.4 | 5.59 | 5.39 | 5.43 | 6.13 | 5.7 | 5.99 | 5.99 | 5.65 | 6.31 | 4.58 | 4.82 | 5.51 |
| D8 | | | | | | | | | | | | | |
| Size | 166.5 | 167.6 | 168.2 | 169.8 | 167.6 | 167 | 172.1 | 170.3 | 173.4 | 170.8 | 168 | 170.3 | 182.3 |
| PdI | 0.09 | 0.09 | 0.1 | 0.097 | 0.094 | 0.081 | 0.074 | 0.113 | 0.139 | 0.068 | 0.136 | 0.113 | 0.121 |
| Content (%) | 5.48 | 99 | 100 | 103 | 99 | 97 | 97 | 99 | 97 | 95 | 92 | 95 | 93 |
| W. content (%) | 5.4 | 5.9 | 5.44 | 5.48 | 5.39 | 5.54 | 5.69 | 5.58 | 5.55 | 6.14 | 4.30 | 4.37 | 4.43 |

1. A powder comprising a plurality of PLGA nanoparticles, each nanoparticle comprising at least one non-hydrophilic material selected from cyclosporine A (Cys A), tacrolimus, pimecrolimus, tetrahydrocannabinol (THC), cannabidiol (CBD), oxaliplatin palmitate acetate (OPA), finasteride, zafirlukast and dexamethasone palmitate; and optionally at least one oil, the powder being in the form of dry flakes prepared by lyophilization from a dispersion comprising said nanoparticles.

2.-3. (canceled)

4. The powder according to claim 1, further comprising at least one cryoprotectant.

5. The powder according to claim 4, wherein the at least one cryoprotectant is selected from cyclodextrin, PVA, sucrose, trehalose, glycerin, dextrose, polyvinylpyrrolidone, xylitol and mannitol.

6. The powder according to claim 1, wherein lyophilization is carried out in the presence of at least one cryoprotectant.

7.-11. (canceled)

12. The powder according to claim 1, wherein the non-hydrophilic material is selected from, tacrolimus and pimecrolimus.

13.-14. (canceled)

15. The powder according to claim 1, wherein the at least one oil comprises castor oil or oleic acid.

16.-22. (canceled)

23. The powder according to claim 1, wherein the non-hydrophilic material is embedded within the nanoparticle polymer.

24. The powder according to claim 1, being a dry powder characterized by one or more of dry of water, free of water, absent of water, substantially dry, comprising no more than 1%-5% water, comprising only water of hydration.

25.-32. (canceled)

33. A reconstituted formulation comprising a powder in a liquid carrier, said powder comprising a plurality of PLGA nanoparticles, each nanoparticle comprising at least one non-hydrophilic material and optionally at least one oil, the powder being in the form of dry flakes prepared by lyophilization from a dispersion comprising said nanoparticles.

34. The formulation according to claim 33, wherein the carrier is water-based or silicone-based.

35.-37. (canceled)

38. The formulation according to claim 33, adapted for oral, enteral, buccal, nasal, topical, transepithelial, rectal, vaginal, aerosol, transmucosal, epidermal, transdermal, dermal, ophthalmic, pulmonary, subcutaneous, intradermal or parenteral administrations.

39.-42. (canceled)

43. The formulation according to claim 33 being an ophthalmic formulation configured for injection or as eye drops.

44.-48. (canceled)

49. A kit comprising a dry lyophilized powder comprising a plurality of PLGA nanoparticles, each nanoparticle comprising at least one non-hydrophilic material, and optionally at least one oil, the powder being in the form of dry flakes prepared by lyophilization from a dispersion comprising said nanoparticles and at least one liquid carrier;
and instructions of use.

50. The kit according to claim 49, wherein the liquid carrier is water or an aqueous solution or an anhydrous (water free) liquid carrier.

51. The formulation according to claim 33, being a pharmaceutical composition for use in a method of treatment of at least one disease or disorder or in a method of delivering at least one non-hydrophilic drug to or across a subject tissue or organ.

52.-60. (canceled)

61. A lyophilized powder comprising PLGA nanoparticles selected from nanocarriers and nanospheres, the nanoparticles comprising at least one agent having a LogP greater than 1, the at least one agent being selected from cyclosporine A (Cys A), tacrolimus, pimecrolimus, dexamethasone palmitate, Cannabis lipophilic extracted derivatives such as tetrahydrocannabinol (THC) and cannabidiol (CBD) (phytocannabinoids), or synthetic cannabinoids, zafirlukast, finasteride and oxaliplatin palmitate acetate (OPA), the powder having a water content not exceeding 7% by weight, relative to the total weight of the powder; wherein said PLGA optionally has an averaged molecular weight of at least about 50 KDa or an averaged molecular weight selected to be different from an averaged molecular weight between 2 and 20 KDa.

62. A dispersion comprising water and a plurality of PLGA nanoparticles selected from nanocarriers and nanospheres, the nanoparticles comprising at least one agent having a LogP greater than 1, the at least one agent being selected from cyclosporine A (Cys A), tacrolimus, pimecrolimus, dexamethasone palmitate, Cannabis lipophilic extracted derivatives such as tetrahydrocannabinol (THC) and cannabidiol (CBD) (phytocannabinoids), or synthetic cannabinoids, zafirlukast, finasteride and oxaliplatin palmitate acetate (OPA), the dispersion being suitable for use within 7 and 28 days; wherein said PLGA optionally has an averaged molecular weight of at least about 50 KDa or an averaged molecular weight selected to be different from an averaged molecular weight between 2 and 20 Kda.

63. A dispersion comprising a silicone carrier and a plurality of PLGA nanoparticles selected from nanocarriers and nanospheres, the nanoparticles comprising at least one agent having a LogP greater than 1, the at least one agent being selected from cyclosporine A (Cys A), tacrolimus, pimecrolimus, dexamethasone palmitate, Cannabis lipophilic extracted derivatives such as tetrahydrocannabinol (THC) and cannabidiol (CBD) (phytocannabinoids), or synthetic cannabinoids, zafirlukast, finasteride and oxaliplatin palmitate acetate (OPA); wherein said PLGA optionally has an averaged molecular weight of at least about 50 KDa or an averaged molecular weight selected to be different from an averaged molecular weight between 2 and 20 KDa.
